(19) United States
(12) Patent Application Publication
(10) Pub. No.: US 2021/0260184 A1
(43) Pub. Date: Aug. 26, 2021

(54) MULTIVALENT CMV VACCINE AND USES THEREOF

(71) Applicant: Duke University, Durham, NC (US)

(72) Inventors: Sallie PERMAR, Durham, NC (US); Cody NELSON, Durham, NC (US); Jennifer JENKS, Durham, NC (US)

(21) Appl. No.: 17/261,554

(22) PCT Filed: Jul. 19, 2019
(86) PCT No.: PCT/US19/42694

§ 371 Date: Jan. 19, 2021

Related U.S. Application Data
(60) Provisional application No. 62/701,606, filed on Jul. 20, 2018.

(51) Int. Cl. A61K 39/295; A61P 31/22; A61K 39/245

(52) U.S. Cl. CPC A61K 39/295; A61P 31/22; A61K 2039/70; C12N 2710/00034; A61K 39/245

(57) ABSTRACT

The invention is directed to multivalent HCMV immunogenic compositions and their use.

This application claims the benefit of priority of U.S. Application Ser. No. 62/701,606 filed Jul. 20, 2018 which content is herein incorporated by reference in its entirety.

This work was supported by NIH/NICHD Director's New Innovator grant DP2HD075699 and fellowship grant F30HD089577. The U.S. Government has certain rights in the invention.

### FIELD OF THE INVENTION

This invention is in the field of cytomegalovirus (CMV) antigens, multivalent composition comprising these CMV antigens and uses of such antigens and compositions to induce immune responses.

### BACKGROUND

Cytomegalovirus is a genus of virus that belongs to the viral family known as Herpesviridae or herpesviruses. The species that infects humans is commonly known as human cytomegalovirus (HCMV) or human herpesvirus-5 (HHV-5). Within Herpesviridae, HCMV belongs to the Betaherpcsvirinac subfamily, which also includes cytomegaloviruses from other mammals.

Though not a well-known cause of birth defects a household name like Zika virus, human cytomegalovirus (HCMV) causes permanent neurologic disability in one newborn child every hour in the United States—more than Down syndrome, fetal alcohol syndrome, and neural tube defects combined.

Although they may be found throughout the body, HCMV infections are frequently associated with the salivary glands. HCMV infects between 50% and 80% of adults in the United States (40% worldwide), as indicated by the presence of antibodies in much of the general population. HCMV infection is typically unnoticed in healthy people, but can be life-threatening for the immunocompromised, such as HIV-infected persons, organ transplant recipients, or new born infants. HCMV is the virus most frequently transmitted to a developing fetus. After infection, HCMV has an ability to remain latent within the body for the lifetime of the host, with occasional reactivations from latency. Given the severity and importance of this disease, obtaining an effective vaccine is considered a public health top priority (See Bernstein D I. 2017. Congenital cytomegalovirus: a “now” problem—no really, now. Clin Vaccine Immunol 24:e00491-16. doi.org/10.1128/CVI.00491-16; Sung, H., et al., (2010) Expert review of vaccines 9, 1303-1314; Schleiss, Expert Opin Ther Pat. April 2010; 20(4): 597-602).

### SUMMARY OF THE INVENTION

There are currently no established effective preventative measures to inhibit congenital HCMV transmission following acute or chronic HCMV infection of a pregnant mother. The gB/MF59 vaccine was only 50% effective at preventing primary infection among young women (Pass, R F, et al., N Engl J Med 2009; 360:1191-9) and yet is the most effective HCMV vaccine tried clinically to date.

While gB is the antigen in several vaccine candidates, in a Phase II clinical trial, the gB/MF59 vaccine was only 50% effective at preventing primary infection among young women with a child at home (Pass, R F, et al., N Engl J Med 2009; 360:1191-9), which is the highest level of protection for any vaccine candidate so far.

Here, we used high-throughput sequencing of viral DNA isolated from patients enrolled in the gB/MF59 vaccine trial to investigate how the vaccine impacted the size, complexity, and composition of the viral population.

In certain aspects, we identified that gB/MF59 vaccine, wherein the gB antigen is of genotype gB1, may only have prevented acquisition of viral variants that are genetically similar to the vaccine strain, accounting for the partial efficacy of the vaccine. These results show that the protective efficacy could be increased with increase of the genetic diversity of the antigen in a multivalent vaccine regimen that would elicit antibody responses with increased genetic breadth.

Despite being a DNA virus, CMV has a tremendous amount of sequence diversity-comparable to HIV and other RNA viruses. See Renzette N, Bhattachajee B, Jensen J D, Gibson L. Kowalik T F (2011) Extensive Genome-Wide Variability of Human Cytomegalovirus in Congenitally Infected Infants. PLoS Pathog 7(5): e1001344.

The inventors' hypothesis is that HCMV infects as a swarm of genetically-distinct variants, and low-frequency intrahost variants are stable over time. For a description of genetic diversity in congenitally infected urine see Renzette N, Gibson L, Bhattacharjee B, Fisher D, Schleiss M R, Jensen J D, et al. (2013) Rapid Intrahost Evolution of Human Cytomegalovirus Is Shaped by Demography and Positive Selection. PLoS Genet 9(9): e1003735.

In certain aspects, the invention provides that minor HCMV variants present at low frequency contribute to diversity in vivo. There are also potential differences in vaccine intrahost viral population including reduced HCMV shedding in saliva, increased compartmentalization at gB locus and a lack of acquisition of gB1 genotype (autologous vaccine strain).

In certain aspects the invention provides that gB immunogen strain-specific protection may have defined vaccine protection. In a non-limiting embodiment, immunogen breadth, for example but not limited to representation of different genotypes, is a consideration in vaccine design.

In certain aspects the invention provides immunogenic compositions against HCMV, wherein the compositions incorporate a plurality of genetically-diverse viral immunogens thereby in certain embodiments inducing responses with increased potency and/or breadth. In some embodiments genetic diversity is represented by different genotypes of the antigen. In certain aspects the invention provides a multivalent vaccine comprising at least one CMV antigen with at least two different CMV genotypes. In certain embodiments, the antigen is gB and the genotype is gB1, gB2, gB3, gB4 and/or gB genotype gB5, or any combination thereof. The gB5 sequence in FIG. 16 shows a new embodiment of a gB5 genotype.

In some embodiments the invention provides HCMV vaccines that include at least one HCMV antigen, e.g. antigenic or an immunogenic fragment or epitope thereof, wherein the antigenic or an immunogenic fragment or epitope thereof is representative of at least two different genotypes. In some embodiments the invention provides HCMV vaccines that include two or more HCMV antigens, e.g. antigenic polypeptides or an immunogenic fragment or epitope thereof, wherein at least one of the antigenic or an immunogenic fragment or epitope thereof is representative of at least two different genotypes. In some embodiments, one of the antigens is gB. In some embodiments the antigens are present at polypeptides. In some embodiments, the antigens are present as nucleic acids. In some embodiments the invention provides HCMV vaccines that include two or more DNA and/or RNA polynucleotides having an open reading frame encoding two or more HCMV antigenic polypeptides or immunogenic fragments or epitopes thereof. The one or more HCMV antigenic polypeptides may be encoded on a single DNA and/or RNA polynucleotide or may be encoded individually on multiple (e.g., two or more) DNA and/or RNA polynucleotides.

In some embodiments, an antigenic polypeptide is an HCMV glycoprotein. For example, a HCMV glycoprotein may be selected from HCMV gH, gL, gB, gO, gN, and gM and an immunogenic fragment or epitope thereof. In some embodiments, the antigenic polypeptide is a HCMV gB polypeptide. Any other suitable HCMV polypeptide could be used, including without limitations CMV protein selected from UL83, UL123, UL128, UL130 and UL131A or an immunogenic fragment or epitope thereof.

The invention provides compositions comprising recombinantly produced protein antigens, nucleic acids, or any combination thereof. In some embodiments the compositions are immunogenic. Methods and animal models to determine immunogenicity of CMV vaccine candidates are known in the art. See e.g. US Pub 20180028645A1, US Pub 20170369532, John et al. in Vaccine Volume 36, Issue 12, 14 Mar. 2018, Pages 1689-1699. In some embodiments the compositions comprise any suitable carrier, adjuvant, or combination thereof.

In some embodiments, wherein the composition comprises recombinant glycoproteins, the glycoprotein may be modified to delete certain domains of the polypeptide. For non-limiting examples of modifications see Pass et al. N Engl J Med 2009; 360:1191-9, and Finnefrock A C et al. Hum Vaccin Immunother. 2016 Aug. 2; 12(8):2106-2112. Epub 2016 Mar. 17, incorporated by reference in their entirety, and FIG. 6. Methods to produce CMV proteins recombinantly are known in the art. See US Pub 20180028645A1, US Pub 20170369532, where the content is incorporated by reference in its entirety.

In some embodiments the antigens are nucleic acids, including but not limited to mRNAs which could be modified and/or unmodified. See US Pub 20180028645A1. US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In some aspects the invention provides vectors comprising the nucleic acids of the invention. In some aspects, the invention provides a host cell, cell cultures or plurality of host cells comprising the nucleic acids of the invention.

In some aspects the invention provides methods of inducing CMV responses using the inventive antigens and compositions of the invention. In some embodiments, the polyvalent vaccination could be achieved using a composition(s) comprising multiple antigens of different genotypes. In some embodiments, the polyvalent vaccination could be achieved using multiple steps of administering composition(s) comprising single antigens of a given genotype. Immunogens of the invention can be administered as proteins, nucleic acids and/or any combination thereof.

The invention provides a composition comprising a polyvalent selection of hCMV antigens or hCMV viruses of different genotypes. In certain aspects the invention provides a multivalent composition comprising at least one human cytomegalovirus (hCMV) antigen or portion thereof, wherein the antigen or portion thereof is represented by at least two different genotypes. In certain embodiments, the composition is immunogenic. In certain embodiments the composition is used in methods to induce antibody responses. In some embodiments, the composition comprises at least two different genotypes, at least three different genotypes, at least four genotypes, at least five genotypes, or any other number of genotypes representing the genetic diversity of the hCMV antigen. In some embodiments, the at least two different genotypes are two different genotypes. In some embodiments, the at least two different genotypes are three different genotypes. In some embodiments, the at least two different genotypes are four different genotypes. In some embodiments, the at least two different genotypes are five different genotypes. In some embodiments, the at least three different genotypes are three different genotypes. In some embodiments, the at least three different genotypes are four different genotypes. In some embodiments, the at least three different genotypes are five different genotypes. In some embodiments, the at least four different genotypes are four different genotypes. In some embodiments, the at least four different genotypes are five different genotypes. In some embodiments, the at least five different genotypes are five different genotypes. If any further antigens or genotypes are identified, the invention contemplates a polyvalent selection comprising combinations of such antigens and/or genotypes. In certain embodiments, the antigen or portion thereof is gB.

In some embodiments the antigen is UL55/gB or a suitable portion thereof. In some embodiments, the two genotypes are any two genotypes of gB antigens. Non-limiting embodiments include: CMV gB1 and CMV gB2, CMV gB1 and CMV gB3, CMV gB1 and CMV gB4, CMV gB1 and CMV gB5, CMV gB2 and CMV gB3, CMV gB2 and CMV gB4, CMV gB2 and CMV gB5, CMV gB3 and CMV gB4, CMV gB3 and CMV gB5, CMV gB4 and CMV gB5, or any combination thereof.

In some embodiments, the three genotypes are any three genotypes of gB antigens. Non-limiting embodiments include: CMV gB1, CMV gB2 and CMV gB3; CMV gB1, CMV gB2 and CMV gB4; CMV gB1, CMV gB2 and CMV gB5; CMV gB2, CMV gB3 and CMV gB4; CMV gB2, CMV gB3 and CMV gB5; CMV gB3, CMV gB4 and CMV gB5, or any combination thereof.

In some embodiments, the four genotypes are any four genotypes of gB antigens. Non-limiting embodiments include: CMV gB1. CMV gB2, CMV gB3 and CMV gB4; CMV gB1, CMV gB2, CMV gB3 and CMV gB5; CMV gB2, CMV gB3, CMV gB4 and CMV gB5; CMV gB1, CMV gB3, CMV gB4 and CMV gB5, or any combination thereof.

In some embodiments, the four genotypes are any five genotypes of gB antigens. Non-limiting embodiments include: CMV gB1. CMV gB2, CMV gB3, CMV gB4 and CMV gB5.

In some embodiments, the antigen is UL55/gB, gH, gL, UL128, UL130, UL131A and/or any other suitable CMV antigen. See 20180028645, or any other suitable antigen from hCMV; see also John et al. Vaccine 36 (2018) 1689-1699.

In some embodiments non-limiting examples of five gB genotypes are shown in FIGS. 15 and 16. See also Murthy et al. supra. at FIGS. 2, 3, 4, and 5 and Table 2 for antigens and genotypes.

In some aspects the invention provides a composition comprising the gB5 polypeptide or nucleic acid sequence of FIG. 16. In some embodiments the composition comprises an antigen of any other gB genotype or any other suitable antigen.

In some aspects the invention provides a multivalent composition comprising at least two hCMV gB polypeptides or nucleic acids encoding gB polypeptides or portions thereof, wherein the gB antigens are of at least two different genotypes, at least three different genotypes, at least four different genotypes, or at least five different genotypes, wherein the genotypes are gB1, gB2, gB2, gB3, gB4, or gB5.

In some embodiments, the at least two different genotypes are: CMV gB1 and CMV gB2, CMV gB1 and CMV gB3, CMV gB1 and CMV gB4, CMV gB1 and CMV gB5, CMV gB2 and CMV gB3, CMV gB2 and CMV gB4, CMV gB2 and CMV gB5, CMV gB3 and CMV gB4, CMV gB3 and CMV gB5, CMV gB4 and CMV gB5, or any combination thereof.

In some embodiments, the at least three different genotypes are: CMV gB1, CMV gB2 and CMV gB3; CMV gB1, CMV gB2 and CMV gB4; CMV gB1, CMV gB2 and CMV gB5; CMV gB2. CMV gB3 and CMV gB4; CMV gB2. CMV gB3 and CMV gB5; CMV gB3, CMV gB4 and CMV gB5, or any combination thereof.

In some embodiments, the at least four different genotypes are: CMV gB1, CMV gB2, CMV gB3 and CMV gB4; CMV gB1, CMV gB2, CMV gB3 and CMV gB5; CMV gB2, CMV gB3, CMV gB4 and CMV gB5; CMV gB1, CMV gB3, CMV gB4 and CMV gB5, or any combination thereof.

In some embodiments, the at least five different genotypes are CMV gB1, CMV gB2, CMV gB3, CMV gB4 and CMV gB5.

In some embodiments, the nucleic acid comprises a modified mRNA.

In certain aspects, the invention provides a composition comprising at least one nucleic acid encoding CMV antigen(s) of at least two different genotypes, at least three different genotypes, at least four different genotypes, or at least five different genotypes, wherein the genotypes of one of the antigens are gB1, gB2, gB2, gB3, gB4, or gB5, wherein the nucleic acid is formulated in at least one lipid nanoparticle (LNP). In some embodiments, the nucleic acid is RNA. In some embodiments, the antigen is CMV gB.

Non-limiting embodiments of nucleic acids and polypeptides of CMV genotypes gB1, gB2, gB2, gB3, gB4, or gB5 are shown in FIGS. 15 and 16. Sequence variants of these genotypes are also contemplated. Various sequence alignment algorithms are known in the art and could be used to determine the genotype of a gB sequence.

In some aspects the invention provides a vector comprising a nucleic acids encoding any one of the antigens of the invention. In some aspects the invention provides a composition comprising the vector.

In some aspects the invention provides a host cells comprising a nucleic acid encoding any one of the antigens of the invention. In some aspects the invention provides a cell culture comprising any of the host cells of the invention.

In some aspects the invention provides methods of inducing an immune response against hCMV comprising administering to a subject in need thereof a composition of the invention.

In some aspects the invention provides methods of inducing immune responses using the compositions of the invention.

### BRIEF DESCRIPTION OF THE DRAWINGS

To conform to the requirements for PCT applications, many of the figures presented herein are black and white representations of images originally created in color.

FIGS. 1A-G. Reduced saliva shedding, yet similar number of viral haplotypes and nucleotide diversity between HCMV-infected glycoprotein B vaccines and placebo recipients. Peak plasma viral load (A) as well as the peak magnitude of virus shed in saliva (B), urine (C), and vaginal fluid (D) was compared between gB vaccines and placebo recipients. Plasma viral load (A) as well as shed virus in urine (C) and vaginal fluid (D) was not statistically different between HCMV-infected placebo recipients and gB/MF59 vaccines, though there was reduced HCMV shedding in the saliva of vaccines (B). The number of unique viral haplotypes (E) as well as nucleotide diversity (π) (F) was assessed in viral DNA amplified at the gB locus for tissue culture virus (TC virus), placebo recipients, gB/MF59 vaccines, and seropositive, chronically HCMV-infected individuals (Sero+). (G) The magnitude of nucleotide diversity resulting in synonymous (π_{S}) vs. nonsynonymous changes (π_{N}) was compared. Horizontal bars indicate the median values for each group. *p<0.05; statistical tests employed include: viral load—Exact Wilcoxon Rank Sum test, haplotypes & π—Kruskal-Wallis test+posthoc Exact Wilcoxon Rank Sum test. π_{S} vs. n_{N}—Wilcoxon Signed Rank test.

FIGS. 2A-D. High magnitude viral shedding in vaginal fluid, yet similar number of unique viral variants and nucleotide diversity between physiologic compartments. Peak viral load was compared between anatomic compartments (A), revealing high-magnitude HCMV shedding in vaginal fluid. The number of unique viral haplotypes (B) as well as nucleotide diversity (π) (C) were defined for viral DNA amplified at the gB locus for tissue culture virus (TCV), as well as virus isolated from whole blood, saliva, urine, and vaginal fluid from acutely-infected gB vaccines and placebo recipients as well as chronically HCMV-infected individuals. (D) The magnitude of nucleotide diversity resulting in synonymous (rrs) vs. nonsynonymous changes (rrN) was compared. Horizontal bars indicate the median values for each group. *p<0.05; statistical tests employed include: viral load—Friedman test+posthoc Pairwise Wilcoxon Signed Rank test, haplotypes & π−Kruskal-Wallis test+posthoc Exact Wilcoxon Rank Sum test, π_{S} vs. π_{N}—Wilcoxon Signed Rank test.

FIGS. 3A-D. Large number of low-frequency viral variants detected in both primary HCMV-infected and chronically-infected individuals. The relative frequency of each unique gB haplotype identified by SNAPP is displayed by individual patient and time point of sample collection. Tissue culture viruses (A) exhibited reduced population complexity by comparison. In primary HCMV-infected placebo recipients (C) and gB vaccines (D), as well as chronically HCMV-infected women (B), there are typically one or more high-frequency haplotypes representing the dominant viral variants within the population which is accompanied by haploptyes at very low frequency representing minor viral variants (<1% of viral haplotype prevalence). Color (colors shown in grayscale per PCT requirements) indicates source fluid: red=blood, blue=saliva, yellow=urine, and pink=vaginal fluid. All haplotypes displayed exceed the 0.44% threshold of PCR and sequencing error established for the SNAPP method (see materials and methods for detail).

FIGS. 4A-D. Evidence of genetic compartmentalization of anatomic compartment-specific viruses at gB locus in 3 of 4 gB vaccines. (A) Table indicating the results of 6 distinct tests of genetic compartmentalization performed on the pool of unique gB haplotypes identified per patient, including Wright's measure of population subdivision (F_{ST}), the nearest-neighbor statistic (Sₙₙ), the Slatkin-Maddison test (SM), the Simmonds association index (AI), and correlation coefficients based on distance between sequences (r) or number of phylogenetic tree branches (r_{b}). For each test, >1000 permutations were simulated. Significant test results suggesting genetic compartmentalization are shown in gray with bold text. Values for F_{ST}, Sₙₙ, SM, r, and r_{b} represent uncorrected p-values, with p<0.05 considered significant. An AI<0.3 was considered a significant result. Three or more positive tests per patient was considered strong evidence for genetic compartmentalization, indicated with a double outline around the result box (originally in green). UP=under-powered (fewer than 5 haplotypes were present in each compartment, making F_{ST} and Sₙₙ error-prone) (B-D) Network of unique viral haplotypes by individual patient, with 1 patient lacking tissue compartmentalization (B) and 2 patients demonstrating strong evidence of viral genetic compartmentalization (D). Samples are organized chronologically from left to right, with blood shown in red, saliva in blue, urine in yellow, and vaginal fluid in purple (colors shown in grayscale per PCT requirements). The size of each node reflects the relative prevalence of each haplotype. Light blue lines connect identical viral variants between time points and compartments, green lines connect variants with a synonymous mutation, and red lines those with a nonsynonymous mutation (colors shown in grayscale per PCT requirements).

FIGS. 5A-C. Possible vaccine immunogen genotype-specific protection in gB/MF59 vaccines. (A) Unrooted phylogenetic tree in a polar layout constructed using full gB open reading frame consensus sequences for each individual sample. Black text indicates placebo recipients, circles are gB/MF59 vaccines, and squares are consensus sequences for each gB genotype. Clades representing gB genotypes are highlighted in different colors and also labelled (colors shown in grayscale per PCT requirements): gB1=purple, gB2=yellow, gB3=blue, gB4=red, and gB5=green. Observed results suggest a possible barrier to acquisition of gB1 genotype viruses following vaccination with a gB1 genotype immunogen (p=0.088; Fischer's Exact test) (B) Number of distinct gB vaccines and placebo recipients with identified consensus viral variants belonging to each gB genotype, inferred by phylogeny. (C) Force-of-infection modeling closely predicts observed gB/MF59 vaccine trial efficacy (Pass et al., N Engl J Med 2009; 360:1191-9). Underlying model assumptions include that gB1 genotype viruses represent 54% of the circulating virus pool and that gB vaccines are universally-protected from acquisition of gB1 viruses.

FIGS. 6A-6B. Linear structure of gB and PCR amplification/next-generation sequencing strategy. (A) The full gB HCMV open reading frame (ORF) is shown, from N-terminus on the left to C-terminus on the right. The four distinct regions of the gB structure are indicated by black bars at the base of the figure, including the ectodomain, membrane proximal external region (MPER), transmembrane domain (TM), and the cytodomain. Major antigenic regions indicated include AD-1 (originally in orange), AD-2 site 1 (originally in red), AD-2 site 2 (originally in yellow), AD-3 (originally in purple), AD-4 (Domain II) (originally in green), and AD-5 (Domain I) (originally in blue). Numbers indicate approximate amino acid residues dividing each region of interest. The gB immunogen employed in this clinical trial contained the full gB ORF with the furin cleavage site mutated and excluding a region from amino acid residue 698 to 773 (containing MPER and TM regions) to facilitate protein secretion during production. Diagram was adapted from Burke H G, Heldwein E E (2015) Crystal Structure of the Human Cytomegalovirus Glycoprotein B. PLoS Pathog 11(10): e1005227 and Chandramouli et al., Nature Communications volume 6, Article number: 8176 (2015). (B) PCR amplification strategy consists of an initial PCR1 step with primers external to the gB ORF, followed by PCR2 amplification of the full gB ORF or an amplicon containing AD-4 and AD-5. Full gB ORF was NGS sequenced to generate a consensus sequence, while gB amplicons were sequenced directly and raw reads used to infer unique viral haplotypes.

FIGS. 7A-B. SNAPP analysis pipeline using SeekDeep. (A) Paired-end reads were obtained for an approximately 550 base-pair amplicon on an Illumina Miseq platform. (B) Paired-end reads were merged, filtered for read quality, then clustered into unique haplotypes. Haplotypes identified in both technical replicates at a frequency above the determined 0.44% cutoff were included for subsequent analysis.

FIGS. 8A-D. UL130 unique viral variants and peak nucleotide diversity is similar both between gB vaccine and placebo groups and between anatomic compartments. The number of unique viral haplotypes as well as nucleotide diversity (π) were assessed for viral DNA amplified at the UL130 locus between treatment groups (A, B) as well as between physiologic compartments (C, D). Tissue culture virus (TC virus), as well as virus isolated from whole blood, saliva, urine, and vaginal fluid. (D) The magnitude of nucleotide diversity resulting in synonymous (rs) vs. nonsynonymous (rrN) changes was compared. Horizontal bars indicate the median values for each group. *p<0.05, viral load=Friedman test+posthoc Pairwise Wilcoxon Signed Rank test, haplotypes & ir=Kruskal-Wallis test+posthoc Exact Wilcoxon Rank Sum test, π_{S} vs. π_{N}=Wilcoxon Signed Rank test.

FIG. 9A-D. Low-frequency viral variants detectable at UL130 locus in both primary HCMV-infected and chronically-infected individuals. The relative frequency of each unique UL130 haplotype identified by SNAPP is displayed by individual patient and time point of sample collection. In primary HCMV-infected placebo recipients (A) and gB vaccines (B) as well as chronically HCMV-infected women (C), there are typically one or more high-frequency haplotypes representing the dominant viral variants comprising the population accompanied by haploptyes at very low frequency representing minor viral variants (<1% of viral haplotype prevalence). Tissue culture viruses (D) exhibited reduced population complexity by comparison. Color indicates source fluid (colors shown in grayscale per PCT requirements): red=blood, blue=saliva, yellow=urine, and pink=vaginal fluid. All haplotypes displayed exceed the 0.44% threshold of PCR and sequencing error established for the SNAPP method (see materials and methods for detail).

FIG. 10. Lack of genetic compartmentalization of anatomic compartment-specific viral variants detected at UL130 locus in vaccines. Table indicating the results of 6 distinct tests for genetic compartmentalization performed on the pool of unique UL130 haplotypes identified per patient, including Wright's measure of population subdivision (F_{ST}), the nearest-neighbor statistic (Sₙₙ), the Slatkin-Maddison test (SM), the Simmonds association index (AI), and correlation coefficients based on distance between sequences (r) or number of phylogenetic tree branches (r_{b}). For each test, >1000 permutations were simulated. Significant test results suggesting genetic compartmentalization are shown in gray with bold text. Values for F_{ST}, Sₙₙ, SM, r, and r_{b} represent uncorrected p-values, with p<0.05 considered significant. An AI<0.3 was considered a significant result. Three or more positive tests per patient was considered strong evidence for genetic compartmentalization, indicated with a double outline around the result box (originally in green).

FIGS. 11A-B. High degree of concordance in haplotype identity and frequency between sequencing replicates. Haplotype identity and frequency were calculated for two technical replicates. The correlation (A) and slope (B) of the haplotype frequencies was compared between technical replicates for both gB and UL130 amplicons, and indicate a high degree of agreement between replicates.

FIGS. 12A-D. Viral load is not correlated with gB antibody-binding or HCMV neutralization. (A) and (B) show that viral load at seroconversion does not correlate with the magnitude of gB-binding (R2=0.0428) (A) nor with HCMV-neutralization titer (R2=0.0035) (B). Furthermore, peak viral load neither correlates with gB-binding (R2=0.0035) (C) nor neutralization titer (R2=0.0009) (D).

FIGS. 13A-B. Higher viral load among women that acquired gB5 genotype viruses. A linear regression analysis of log 10 viral load on genotype was performed at time of seroconversion (A) as well as peak viral load (B). At time of seroconversion, the viral load among women who acquired a gB5 genotype virus was 3.44 time greater than that of women shedding non-gB5 genotype virus (95% CI 1.13-10.51, p=0.031). Solid line for each grouping indicates mean value, whereas dotted black line indicates threshold of qPCR detection (100 copies/mL).

FIGS. 14A-B. Correlations between viral load, number of unique variants, and nucleotide diversity. Kendall Tau correlation coefficients are shown for viral load (VL), number of haplotypes, nucleotide diversity (π), as well as synonymous nucleotide diversity (π_{S}), and nonsynonymous nucleotide diversity (π_{N}). Bold values indicate a significant correlation (uncorrected p<0.05).

FIG. 15 shows non-limiting embodiments of gB nucleic acid sequences of five different genotypes. Genotypes 1-4 were taken from Chou, Virology, 188 (1), 388-390, 1992 (PMID: 1314465). The accessioning number is given in the sequence header. Genotype 5 is a novel sequence from the patient population of the instant study.

FIG. 16 shows non-limiting embodiments of gB amino acid sequences of five different genotypes. Translation of the nucleic acid sequences of FIG. 15.

FIGS. 17A-B shows that mRNA LNP elicits comparable gB binding to protein/adjuvant. Median (A) and range (B) binding to gB was quantified by plate-based ELISA. Area under the curve (AUC) is indicated on the y-axis. Vaccine doses were given at 0, 4, and 8 weeks. Blue=IM Sanofi gB+Addavax adjuvant, green=IM gB ectodomain+Addavax adjuvant, purple=ID gB mRNA LNP. Vaccine doses were 20 ug protein or 50 ug mRNA LNP.

FIGS. 18A and 18B. In two historical cohorts of HCMV-seronegative adolescent and postpartum women vaccinated with HCMV gB1 with MF59 adjuvant, regression analyses revealed that protection against HCMV primary acquisition in vaccines was associated with magnitude IgG binding to gB-transfected cells and IgG binding to gB Domains I+II. Analysis of 23 total vaccine-elicited antibody binding and functional responses in 42 women from the adolescent (14 infected, 28 uninfected) and 33 from the postpartum cohorts (11 infected, 22 uninfected) were measured. Multiple linear regression controlling for cohort was performed for the combined log-transformed group data (apriori significance cut-off of p <0.05, Benjamin-Hochberg FDR<0.2). IgG binding to gB genotype 1-transfected cells (FIG. 18A) was associated with protection against HCMV infection for combined cohorts (p=0.006, FDR=0.15), as well as in subgroup analyses of adolescent (p=0.057, π_{S}) and postpartum (p=0.045) populations. IgG binding to gB Domains 1+11 (FIG. 18B) by Luminex assay was significantly associated with protection in the combined cohorts (p=0.046, FDR 0.15).

FIGS. 19A-D. Vaccination with gB genotype 1 mRNA vaccination elicits more durable binding antibody responses against cell-associated gB than vaccination with gB (Sanofi) protein or gB ectodomain protein. Groups of juvenile New Zealand White rabbits (π=6) were administered 3 sequential doses of gB/MF59 protein IM, gB ectodomain protein (lacking AD-3)+MF59 IM, or lipid nanoparticle (LNP)-packaged gB-encoding nucleoside-modified mRNA ID. All vaccines were highly immunogenic with similar kinetics and comparable peak gB-binding/functional antibody responses. Yet, both ectodomain and mRNA-LNP-immunized rabbits exhibited enhanced durability of gB antibody-binding (p=0.04 and 0.02, respectively), and the mRNA-LNP group had more durable binding of membrane-associated gB (p<0.001).

FIGS. 20A and 20B. HCMV glycoprotein B (gB)-specific monoclonal antibodies isolated from plasmablasts from 3 HCMV-seropositive subjects showed differential binding to cell-associated gB genotypes 1 to 5. 293T cells were transfected with gB mRNA encoding genotypes 1, 2, 3, 4, or 5, then binding of 26 mAbs (from Merck & Co., Inc.) to each gB genotype was measured by flow cytometry. (a) Across most mAbs, mAb binding to gB was measured highest against genotypes 2 and 4. (b) Normalization of gB genotype-specific binding to 100% showed that each mAb differentially recognized the 5 gB genotypes as expressed on transfected cells. In both (A) and (B) the genotypes in each bar are listed in the same order as the genotypes listed on the right side of the figure.

FIG. 21 shows one embodiment of a vaccination schedule for a vaccine immunogenicity study of mRNA-based gB vaccines in rabbits. In an upcoming study, 18 rabbits will be classified into 3 treatment groups and will receive ID injections on Weeks 0, 2, and 4. Each vaccine contains a total 50 μg gB mRNAs encapsulated in lipid nanoparticles (LNP). Treatments groups are as follows: (1) 50 μg gB1 mRNA (π=6), (2) 25 μg gB1 mRNA+25 gB2 mRNA (π=6), and (3) 10 μg gB1 mRNA+10 μg gB2 mRNA+10 μg gB3 mRNA+10 μg gB4 mRNA+10 μg gB5 mRNA. Blood samples will be collected every 2 weeks before week 12, then every 4 week until necropsy at week 24.

FIG. 22. Human epithelial cells transfected with mRNA encoding HCMV gB genotypes 2 to 5 express gB. 293T cells were transfected with HCMV gB mRNAs variants 2, 3, 4, and/or 5 using Mirus Bio Kit according to manufacturer's instructions. mRNAs were transfected individually (1 ug/ml) or in combination (0.5 ug/ml per mRNA). At 20 hours post-transfection, cells were harvested, then cytosolic components were separated using the Mem-PER Plus membrane protein extraction kit (Thermo), in the presence of protease inhibitors. Cytosolic fractions were run on a non-reduced gel, then stained with a gB AD2 mAb (TRL100) at 1:2000 overnight at 4 degrees C. then stained with anti-human secondary at 1:5,000 for 2.5 hours at room temperature and developed. HCMV gB protein (Sanofi) was run as a positive control.

### DETAILED DESCRIPTION

The genomes of over 20 different HCMV strains have been sequenced, including those of both laboratory strains and clinical isolates. For example, the following strains of HCMV have been sequenced: Towne (GL239909366), AD169 (GI:219879600), Toledo (GL290564358) and Merlin (GI: 155573956). HCMV strains AD169, Towne and Merlin can be obtained from the American Type Culture Collection (ATCC VR538, ATCC VR977 and ATCC VR1590, respectively).

Cytomegalovirus contains a number of membrane protein complexes. Of the approximately 30 known glycoproteins in the viral envelope, gH and gL have emerged as particularly interesting due to their presence in several different complexes: dimeric gH/gL, trimeric gH/gL/gO (also known as the gCIII complex), and the pentameric gH/gL/pUL128/pUL130/pUL131 (pUL131 is also referred to as “pUL131A”, “pUL131a”, or “UL131A”; pUL128, pUL130, and pUL131 subunits sometimes are also referred as UL128, UL130, UL131). CMV is thought to use the pentameric complexes to enter epithelial and endothelial cells by endocytosis and low-pH-dependent fusion but it is thought to enter fibroblasts by direct fusion at the plasma membrane in a process involving gH/gL or possibly gH/g/gO. The gH/gL and/or gH/gL/gO complex(es) is/are sufficient for fibroblast infection, whereas the pentameric complex is required to infect endothelial and epithelial cells.

The pentameric complex is considered as a major target for CMV vaccination. See US Pub 20180028645A1. Viral genes UL128, UL130 and UL131 are needed for endothelial entry (Hahn, Journal of Virology 2004; 78:10023-33). Fibroblast-adapted non-endothelial tropic strains contain mutations in at least one of these three genes. Towne strain, for example, contains a two base pair insertion causing a frame shift in UL130 gene, whereas AD169 contains a one base pair insertion in UL131 gene. Both Towne and AD169 could be adapted for growth in endothelial cells, and in both instances, the frame shift mutations in UL130 or UL131 genes were repaired.

U.S. Pat. No. 7,704,510 discloses that pUL131A is required for epithelial cell tropism. U.S. Pat. No. 7,704,510 also discloses that pUL128 and pUL130 form a complex with gH/gL, which is incorporated into virions. This complex is required to infect endothelial and epithelial cells but not fibroblasts. Anti-CD46 antibodies were found to inhibit HCMV infection of epithelial cells.

CMV vaccines tested in clinical trials include Towne vaccine. Towne-Toledo chimeras, an alpha virus replicon with gB as the antigen, gB/MF59 vaccine (see Pass, R F, et al., N Engl J Med 2009; 360:1191-9), a gB vaccine produced by GlaxoSmithKline, and a DNA vaccine using gB and pp65 (See Tang et al. Hum Vaccin Immunother. 2017 Dec. 2; 13(12):2763-2771. doi: 10.1080/21645515.2017.1308988. Epub 2017 May 11). pp65 is viral protein that is a potent inducer of CD8+ responses directed against CMV. These vaccines are all poor inducers of antibodies that block viral entry into endothelial/epithelial cells (Adler, S. P. (2013), British Medical Bulletin, 107, 57-68. doi:10.1093/bmb/Idt023).

Preclinical animal studies in CMV vaccines include an inactivated AD169 which has been repaired in the UL131 gene, a DNA vaccine using a wild-type UL130 gene and peptide vaccines using peptides from pUL130 and 131 (Sauer, A, et al., Vaccine 2011; 29:2705-1, doi:10.1016).

A recombinant Human Cytomegalovirus (HCMV) gL protein vaccine candidate is provided in US Pub 20170369532.

Glycoprotein B (gB) is a homotrimer protein, 907 amino acids in length. It is relatively conserved among herpesviruses and is essential for entry into all cells. Many gB-specific antibodies are neutralizing, though the majority are non-neutralizing. See Burke H G, Heldwein E E (2015) Crystal Structure of the Human Cytomegalovirus Glycoprotein B. PLoS Pathog 11(10): e1005227.

However, there still remains a need for a safe and effective CMV vaccine.

Genetic variability of CMV is well known, with well documented different strains, genotypes and subtypes. See Murthy et al. (2011) Detection of a Single Identical Cytomegalovirus (CMV) Strain in Recently Seroconverted Young Women. PLoS ONE 6(1): e15949. doi.org/10.1371/journal.pone.0015949. Numerous studies have examined a potential link between CMV virulence and pathogenesis, particularly in the context of solid organ transplantation, and sequence polymorphism of the viral genome. See e.g. Pang et al. Am J Transplant. 2009 February; 9(2):258-68. doi: 10.1111/j.1600-6143.2008.02513.x., without finding of clear correlation between sequence polymorphism and viral pathogenesis, see Sarcinella et al. J Clin Virol. 2002 February; 24(1-2):99-105, Humar et al. J Infect Dis. 2003 Aug. 15; 188(4):581-4. Epub 2003 July 29.

As disclosed and exemplified herein, analyzing gB/MIF59 vaccines and placebo recipient samples (see Example 1), the inventors have discovered an enrichment of gB1 genotype HCMV variants among placebo recipients (7/13 placebo recipients vs. 0/5 gB vaccines), which suggests that the gB1 genotype vaccine antigen may have elicited genotype-specific protection. These data indicate that gB immunization may have had a measurable impact on viral intrahost population dynamics. These finding suggest that including multiple HCMV genotypes in a multivalent vaccine composition may achieve breadth of HCVM protection.

CMV genetic diversity is known and characterized. For a discussion of CMV genotypes see e.g. Murthy et al. (2011) Detection of a Single Identical Cytomegalovirus (CMV) Strain in Recently Seroconverted Young Women. PLoS ONE 6(1): c15949. doi.org/10.1371/journal.pone.0015949: Pang et al. Am J Transplant. 2009 February; 9(2):258-68. doi: 10.1111/j.1600-6143.2008.02513.x., specifically reference to Chou and Dennison at second paragraph in the introduction: Coaguette et al. Clin Infect Dis. 2004 Jul. 15; 39(2):155-61. Epub 2004 Jun. 23, specifically at discussion and references in second paragraph in introduction, all of which content is incorporated by reference in its entirety.

In some embodiments, the antigens are administered as recombinant proteins. In other embodiments the antigens are administered as nucleic acids. Non-limiting embodiments include mRNA, including but not limited to modified mRNA. See e.g. US Pub 20180028645A1.

Genetic variability of CMV antigens can be determined by those of ordinary skill in the art by obtaining sequence data and aligning the amino acid and/or nucleic sequences using readily available and well-known alignment algorithms (such as BLAST, using default settings; ClustalW2; or algorithm disclosed by Corpet, Nucleic Acids Research, 1998, 16(22):10881-10890).

Pharmaceutical Compositions and Administration

In certain aspects the invention provides pharmaceutical compositions comprising the CMV proteins and nucleic acids of the invention.

The diverse CMV proteins and nucleic acids described herein can be incorporated into an immunogenic composition, or a vaccine composition. Such compositions can be used to raise antibodies in a mammal (e.g. a human).

The invention provides pharmaceutical compositions comprising the CMV proteins and nucleic acids described herein, and processes for making a pharmaceutical composition involving combining the CMV proteins and nucleic acids described herein with a pharmaceutically acceptable carrier. The pharmaceutical compositions of the invention typically include a pharmaceutically acceptable carrier, and a thorough discussion of such carriers is available in Remington: The Science and Practice of Pharmacy.

The pH of the composition is suitable for physiological use, and is usually between about 4.5 to about 11. Stable pH may be maintained by the use of a buffer e.g. a Tris buffer, a citrate buffer, a phosphate buffer, or a histidine buffer. Thus a composition will generally include a buffer.

A composition may be sterile and/or pyrogen free. Compositions may be isotonic with respect to humans.

A composition comprises an immunologically effective amount of its antigen(s). A skilled artisan can readily determine the effective amount.

Immunogenic compositions may include an immunological adjuvant. Exemplary adjuvants include mineral-containing compositions; oil emulsions; saponin formulations; virosomes and virus-like particles; bacterial or microbial derivatives; bioadhesives and mucoadhesives; liposomes; polyoxyethylene ether and polyoxyethylene ester formulations; polyphosphazene (pcpp); muramyl peptides; imidazoquinolone compounds; thiosemicarbazone compounds; tryptanthrin compounds; human immunomodulators; lipopeptides; benzonaphthyridines; microparticles: immunostimulatory polynucleotide (such as RNA or DNA: e.g., CpG-containing oligonucleotides).

For example, the composition may include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design (1995) eds. Powell & Newman. ISBN: 030644867X. Plenum), or mixtures thereof. The salts can take any suitable form (e.g. gel, crystalline, amorphous, etc.), with adsorption of antigen to the salt being an example.

One suitable immunological adjuvant comprises a compound of Formula (I) as defined in WO2011/027222, or a pharmaceutically acceptable salt thereof, adsorbed to an aluminum salt. Many further adjuvants can be used, including any of those disclosed in Powell & Newman (1995).

Compositions may include an antimicrobial, particularly when packaged in multiple dose format. Antimicrobials such as thimerosal and 2 phenoxyethanol are commonly found in vaccines, but sometimes it may be desirable to use either a mercury-free preservative or no preservative at all.

Compositions may comprise detergent e.g. a polysorbate, such as polysorbate 80. Detergents are generally present at low levels e.g. <0.01%.

Compositions may include sodium salts (e.g. sodium chloride) to give tonicity. A concentration of 10+−2 mg/ml NaCl is typical, e.g., about 9 mg/ml.

In certain embodiments, where the composition comprises nucleic acids such as mRNA, whether modified or unmodified, the mRNAs could be formulated in lipid nanoparticles (LNPs). See US Pub 20180028645A1 and WO 2015/164674), the content of which is incorporated by reference in its entirety.

In another aspect, the invention provides a method of inducing an immune response against cytomegalovirus (CMV), comprising administering to a subject in need thereof an immunologically effective amount of the immunogenic composition, which comprises the proteins, DNA molecules, RNA molecules (e.g., self-replicating RNA molecules, mRNAs), or virus-like replicon particles (VRPs) as described herein. In certain embodiments the compositions are administered to provide protection against congenital CMV infection. In certain embodiments the compositions are administered to provide protection against CMV infection in immunocompromised individuals, transplant recipients, or during organ transplantation.

In certain embodiments, the immune response comprises the production of neutralizing antibodies against CMV. In certain embodiments, the neutralizing antibodies are complement-independent.

The immune response can comprise a humoral immune response, a cell-mediated immune response, or both. In some embodiments an immune response is induced against each delivered CMV protein. A cell-mediated immune response can comprise a Helper T-cell (Th) response, a CD8+ cytotoxic T-cell (CTL) response, or both. In some embodiments the immune response comprises a humoral immune response, and the antibodies are neutralizing antibodies. Neutralizing antibodies block viral infection of cells. CMV infects many cell types, including epithelial cells and also fibroblast cells. In some embodiments the immune response reduces or prevents infection of both cell types. Neutralizing antibody responses can be complement-dependent or complement-independent. In some embodiments the neutralizing antibody response is complement-independent. In some embodiments the neutralizing antibody response is cross-neutralizing; i.e., an antibody generated against an administered composition neutralizes a CMV virus of a strain other than the strain used in the composition.

Compositions of the invention will generally be administered directly to a subject. Direct delivery may be accomplished by parenteral injection (e.g. subcutaneously, intraperitoneally, intravenously, intramuscularly, or to the interstitial space of a tissue), or by any other suitable route. For example, intramuscular administration may be used e.g. to the thigh or the upper arm. Injection may be via a needle (e.g. a hypodermic needle), but needle-free injection may alternatively be used. A typical intramuscular dosage volume is about 0.5 ml.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

The subject may be an animal, preferably a vertebrate, more preferably a mammal. Exemplary subject includes, e.g., a human, a cow, a pig, a chicken, a cat or a dog, as the pathogens covered herein may be problematic across a wide range of species. Where the vaccine is for prophylactic use, the human is preferably a child (e.g., a toddler or infant), a teenager, or an adult; where the vaccine is for therapeutic use, the human is preferably a teenager or an adult. A vaccine intended for children may also be administered to adults, e.g., to assess safety, dosage, immunogenicity, etc.

Vaccines of the invention may be prophylactic (i.e. to prevent disease) or therapeutic (i.e. to reduce or eliminate the symptoms of a disease). The term prophylactic may be considered as reducing the severity of or preventing the onset of a particular condition. For the avoidance of doubt, the term prophylactic vaccine may also refer to vaccines that ameliorate the effects of a future infection, for example by reducing the severity or duration of such an infection.

Isolated and/or purified CMV proteins, complexes, and nucleic acids described herein can be administered alone or as either prime or boost in mixed-modality regimes, such as a RNA prime followed by a protein boost. Benefits of the RNA prime protein boost strategy, as compared to a protein prime protein boost strategy, include, for example, increased antibody titers, a more balanced IgG1:IgG2a subtype profile, induction of TH1-type CD4+ T cell-mediated immune response that was similar to that of viral particles, and reduced production of non-neutralizing antibodies. The RNA prime can increase the immunogenicity of compositions regardless of whether they contain or do not contain an adjuvant.

In the RNA prime-protein boost strategy, the RNA and the protein are directed to the same target antigen. Examples of suitable modes of delivering RNAs include virus-like replicon particles (VRPs), alphavirus RNA, replicons encapsulated in lipid nanoparticles (LNPs) or formulated RNAs, such as replicons formulated with cationic nanoemulsions (CNEs). Suitable cationic oil-in-water nanoemulsions are disclosed in WO2012/006380 e.g. comprising an oil core (e.g. comprising squalene) and a cationic lipid (e.g. DOTAP, DMTAP, DSTAP, DC-cholesterol, etc.).

An RNA prime-protein boost regimen may involve first (e.g. at weeks 0-8) performing one or more priming immunization(s) with RNA (which could be delivered as VRPs, LNPs, CNEs, etc.) that encodes one or more of the protein components of a CMV protein complex of the invention and then perform one or more boosting immunization(s) later (e.g. at weeks 24-58) with: an isolated CMV protein complex of the invention, optionally formulated with an adjuvant or a purified CMV protein complex of the invention, optionally formulated with an adjuvant. In some embodiments, the prime and boost vaccine comprise the same immunogen(s). In some embodiments, the prime and boost vaccine comprise difference immunogens.

In some embodiments, the RNA molecule is encapsulated in, bound to or adsorbed on a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion, or combinations thereof.

Also provided herein are kits for administration of nucleic acid (e.g., RNA), purified proteins, and purified nucleic acids described herein, and instructions for use. The invention also provides a delivery device pre-filled with a composition or a vaccine disclosed herein.

The pharmaceutical compositions described herein can be administered in combination with one or more additional therapeutic agents. The additional therapeutic agents may include, but are not limited to antibiotics or antibacterial agents, antiemetic agents, antifungal agents, anti-inflammatory agents, antiviral agents, immunomodulatory agents, cytokines, antidepressants, hormones, alkylating agents, antimetabolites, antitumor antibiotics, antimitotic agents, topoisomerase inhibitors, cytostatic agents, anti-invasion agents, antiangiogenic agents, inhibitors of growth factor function inhibitors of viral replication, viral enzyme inhibitors, anticancer agents, .alpha.-interferons, .beta.-interferons, ribavirin, hormones, and other toll-like receptor modulators, immunoglobulins (Igs), and antibodies modulating Ig function (such as anti-IgE (omalizumab)).

In certain embodiments, the compositions disclosed herein may be used as a medicament, e.g., for use in inducing or enhancing an immune response in a subject in need thereof, such as a mammal.

In certain embodiments, the compositions disclosed herein may be used in the manufacture of a medicament for inducing or enhancing an immune response in a subject in need thereof, such as a mammal.

One way of checking the efficacy of therapeutic treatment involves monitoring pathogen infection after administration of the compositions or vaccines disclosed herein. Another way of checking the efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

This invention is further illustrated by the following examples which should not be construed as limiting.

### EXAMPLES

### Example 1

Example 1: Genetic signatures of human cytomegalovirus variants acquired by seronegative glycoprotein B vaccines

Human cytomegalovirus (HCMV) is the most common congenital infection worldwide, and a frequent cause of hearing loss or debilitating neurologic disease in newborn infants. Thus, a vaccine to prevent HCMV acquisition is a public health priority. The glycoprotein B (gB)+MF59 adjuvant subunit vaccine is the most efficacious tested clinically to date, demonstrating approximately 50% efficacy in multiple phase 2 trials. Yet, the impact of gB/MF59-elicited immune responses on the population of viruses acquired by trial participants has not been assessed. In this analysis, we employed quantitative PCR as well as two distinct next-generation sequencing strategies (short amplicon and whole gene) to interrogate genetic differences between the HCMV populations infecting gB/MF59 vaccines and placebo recipients. For the majority of subject-specific viral populations analyzed, we identified a single dominant genetic haplotype along with a large number of minor haplotypes present at low frequency. This finding suggests that the intrahost viral population constitutes a heterogeneous swarm of genetically-distinct viral variants. Additionally, we identified several possible distinctions between the viral populations of acutely-infected vaccines and placebo recipients. First, there was reduced magnitude peak viral shedding in the saliva of gB vaccines compared to placebo (median copies/mL: placebo=319, gB/MF59=51; p=0.02, Exact Wilcoxon Rank Sum test). Furthermore, employing a panel of tests for genetic compartmentalization, we noted evidence of tissue-specific gB haplotypes in 3 of 4 vaccines, though only in 1 of 7 placebo recipients. Finally, we observed an enrichment of gB1 genotype HCMV variants among placebo recipients (7/13 placebo recipients vs. 0/5 gB vaccines), which suggests that the gB1 genotype vaccine antigen may have elicited genotype-specific protection. These data indicate that gB immunization may have had a measurable impact on viral intrahost population dynamics and support future analysis of a larger cohort.

Human cytomegalovirus (HCMV) congenital infection affects 1 in 150 pregnancies (1) and is the most frequent non-genetic cause of sensorineural hearing loss and neurodevelopmental delay in infants worldwide (2). Additionally, HCMV is the most common infectious agent among allograft recipients, often causing end-organ disease such as hepatitis, pneumonitis, or gastroenteritis and predisposing the individual to graft rejection (3). It has been estimated that an efficacious HCMV vaccine would save the United States 4 billion dollars and 70,000 quality-adjusted life years annually, and thus HCMV vaccine development has remained a tier 1 priority of the National Academy of Medicine for the past 17 years (4).

The glycoprotein B (gB)+MF59 adjuvant vaccine is the most efficacious HCMV vaccine platform trialed to date, demonstrating partial vaccine protection in multiple patient populations. In a cohort of HCMV-seronegative postpartum women, gB vaccination achieved a promising 50% vaccine efficacy (5). When this study was subsequently repeated in a cohort of seronegative adolescent women, a comparable level of vaccine-protection was observed (6). Furthermore, in allograft recipients, the same gB vaccine reduced duration of HCMV viremia and antiviral therapy (7). The mechanism of this partial vaccine protection remains unknown, though we and others have observed this vaccine platform was particularly poor at eliciting heterologous neutralizing antibodies in these populations and that non-neutralizing antibody responses may have played a role. See Nelson et al. Proc Natl Acad Sci USA. 2018 Apr. 30. pii: 201800177. Yet any distinction between viruses acquired by gB/MF59 vaccines and placebo recipients has not been thoroughly evaluated, and this remains a critical question for understanding the functional antiviral immunity responsible for the partial vaccine efficacy demonstrated in these clinical trials.

With a genome consisting of 236 kilobase pairs (8) and encoding approximately 164 open reading frames (9), HCMV has the largest genome of any human virus. Thus, prior to the advent of whole-genome sequencing, it was extraordinarily challenging to assess HCMV viral population composition and diversity because of the limitations of traditional sequencing methodologies. Nevertheless, it is now well established that HCMV is highly polymorphic between and within individuals, defined via a variety of sequencing methodologies including restriction fragment length polymorphism analysis (10), targeted gene sequencing (11-16), and whole genome sequencing (17-19). Yet the source of this diversity remains poorly understood. If multiple unique viral variants are identified in a single individual (so-called “mixed infection”), does this represent de novo mutations, simultaneous initial infection with multiple unique variants, or independent, sequential infection events?Mixed infections have been frequently detected in both chronically HCMV-infected individuals (20) and immunocompromised hosts (12-14). Yet, recently-seroconverted women from the gB/MF59 vaccine trial predominantly had a single variant detected in all tissues and at all time points (16) when evaluated by a traditional Sanger sequencing methodology, suggesting that mixed infections in healthy individuals may result from independent, sequential infection events.

One major limitation of traditional HCMV genotyping to quantify HCMV diversity is a lack of sensitivity to detect viral variants present at low frequency. Recent HCMV whole-genome, next-generation sequencing (NGS) has suggested that there is remarkable intrahost diversity, comparable to many RNA viruses, stemming from the presence of low-frequency alleles representing minor viral variants (17-19). Thus, it has been established that HCMV likely exists within individual hosts as a heterogeneous population of unique, but related viral variants (19). Subsequent characterization of the intrahost composition and distribution of low-frequency viral variants, has led to recognition of unique viral populations between individual organs representing anatomic compartmentalization of viral populations (18).

As HCMV diversity is due to the presence of distinct, low-frequency viral variants, traditional sequencing methodologies may not be the most appropriate means to discern differences between intrahost viral populations. Thus, we applied a previously-validated (21) sequencing methodology and analysis pipeline termed Short NGS Amplicon Population Profiling (SNAPP) to investigate the viral populations of recently-seroconverted gB/MF59 vaccines and placebo recipients. This technique, which employs sequencing of an approximately 500 base-pair region at tremendous read depth, has facilitated a more complete understanding of in vivo viral dynamics. We hypothesize that gB/MF59 vaccination limited the complexity of the in vivo viral population following primary HCMV infection. Metrics of HCMV population dynamics including viral load, pairwise genetic diversity, number of unique haplotypes (viral variants), and the characteristics of those variants can be studied in concert with vaccine-elicited immune responses to arrive at a more comprehensive understanding of the mechanism of partial vaccine efficacy.

Results:

Viral Load, Number of Haplotypes, and Haplotype Sequence Diversity by Vaccine Group

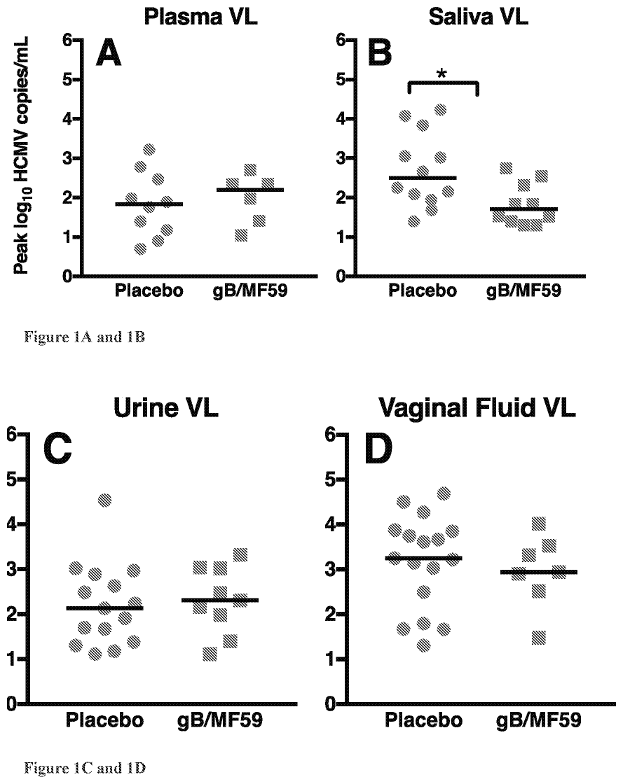

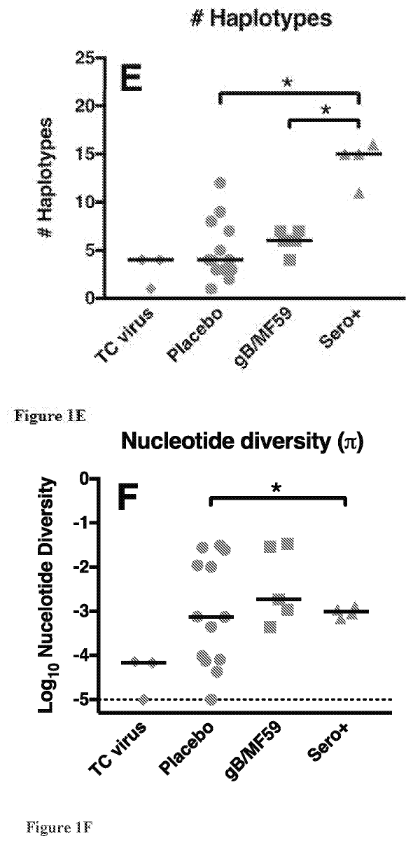

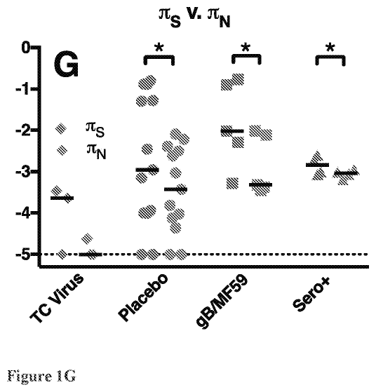

We obtained HCMV DNA extracted from plasma, saliva, urine, and vaginal fluid of 53 gB/MF59 vaccine and placebo recipients following HCMV primary infection. Samples were taken approximately monthly, though sampling was heterogeneous between trial participants. Peak plasma viral load (FIG. 1A) and peak viral shedding in saliva, urine, and vaginal fluid (FIG. 1B-D) was identified for each patient and separated by vaccine group. The peak levels of viremia following primary HCMV acquisition were not significantly different between vaccine and placebo recipients. The viral load of shed virus in urine and vaginal fluid was also not significantly different between placebo and gB/MF59 vaccines. Levels of saliva viral shedding, however, were reduced in gB vaccines (median copies/mL: Pb=319, gB/MF59=51; p=0.022, Friedman test+poshoc Exact Wilcoxon Rank Sum test).

Next, short (˜550 base pair), variable regions within gB and UL130 (membrane glycoprotein targeted by neutralizing antibodies, but not included in gB/MF59 vaccine) were amplified by nested PCR then deep-sequenced (FIG. S1). Unique viral haplotypes were inferred by a modified SeekDeep analysis pipeline (22) (FIG. S2). gB and UL130 haplotypes were obtained from a total of 14 placebo-recipients and 6 gB/MF59 vaccines following primary infection, as well as 4 seropositive, chronically HCMV-infected individuals. Three tissue culture virus stocks were included as a genetically-homogenous comparison. The peak number of viral haplotypes among all compartments for each patient was similar between placebo and gB/MF59 vaccines following primary HCMV infection at both the gB (FIG. 1E) and UL130 loci (FIG. S3A). Interestingly, the peak number of gB viral haplotypes was higher in chronically-infected seropositive individuals compared to both placebo (median haplotypes: SP=15, P=4; p=0.002, Kruskal-Wallis test+posthoc Exact Wilcoxon Rank Sum test) and gB/MF59 vaccines (median haplotypes: gB/MF59=6; p=0.008, Kruskal-Wallis+Exact Wilcoxon Rank Sum test). However, this trend was not identified at the UL130 locus.

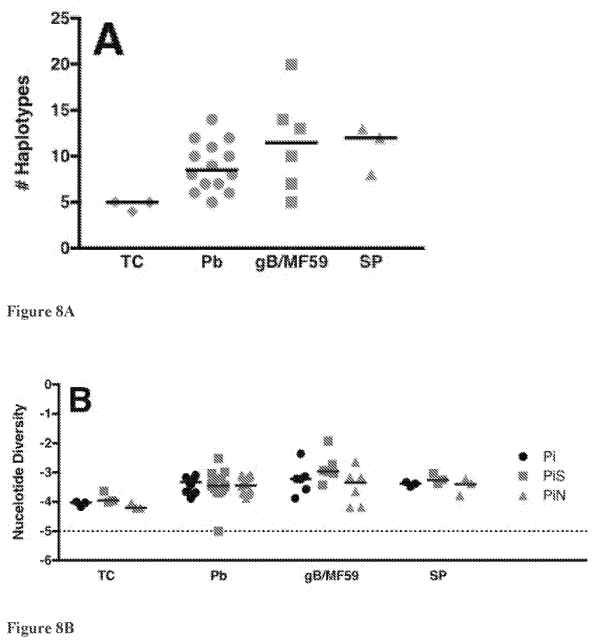

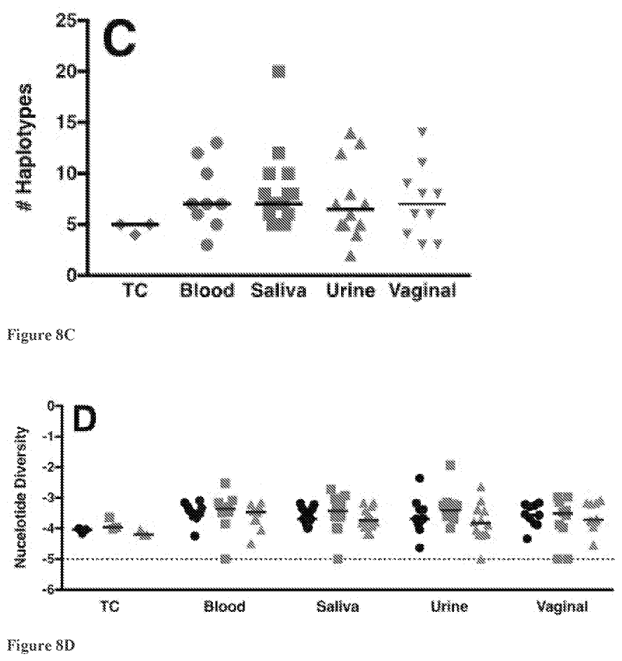

The peak nucleotide diversity (π) for each patient was calculated for identified haplotypes at the gB (FIG. 1F) and UL130 loci (FIG. 8B). There was no statistical difference in gB (π) between placebo recipients and vaccines following primary HCMV infection, though seropositive individuals had higher gB (π) in comparison to primary HCMV-infected placebo recipients (median (π): SP=9.8×10⁻⁴, Pb=7.3×10⁻⁴; p=0.011, Kruskal-Wallis test+posthoc Exact Wilcoxon Rank Sum test). π attributable to synonymous mutations ((π_{S}) and nonsynonymous mutations (π_{N}) was further compared within each group at the gB (FIG. 1G) and UL130 (FIG. S3B) loci. Again, there was no difference in π_{S} or π_{N} between infected placebo and vaccine recipients. However, the gB π_{S} significantly exceeded N for primary HCMV-infected placebos (p=0.004, Wilcoxon Signed Rank test) and gB vaccines (p=0.001, Wilcoxon Signed Rank test) as well as for seropositive, chronically HCMV-infected individuals (p=0.016, Wilcoxon Signed Rank test), indicating that purifying selection was pervasive in the viral populations of each of these groups. Of note, the enhanced nucleotide diversity of seropositive individuals over acutely-infected placebo recipients was not identified at the UL130 locus. In UL130, π_{S} was also only greater than π_{N} in the gB/MF59 vaccine subgroup (p=0.006, Wilcoxon Signed Rank test). Overall, these data suggest that there the genetic complexity of the viral population in acutely-infected vaccines vs placebo recipients is similar, though reduced compared to the viral population in chronically HCMV-infected individuals.

Viral Load, Number of HCMV Haplotypes, and Sequence Diversity by Anatomic Compartment.

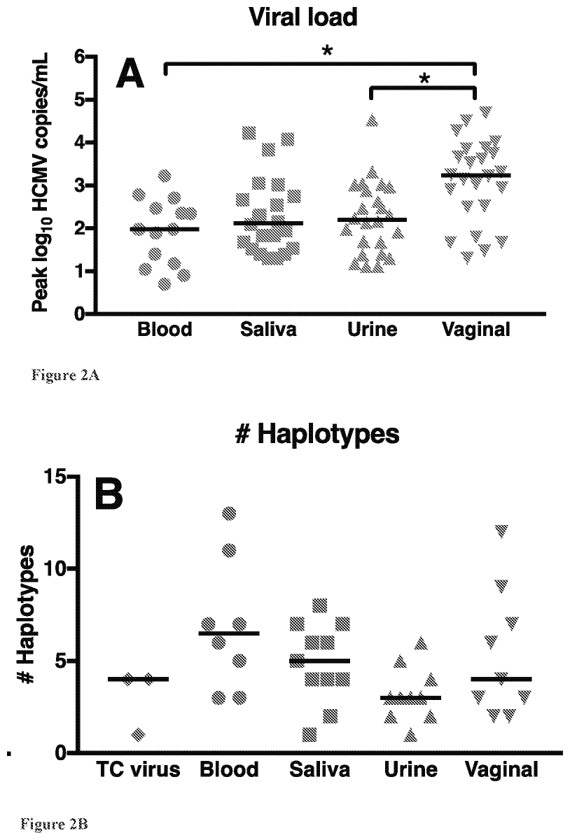

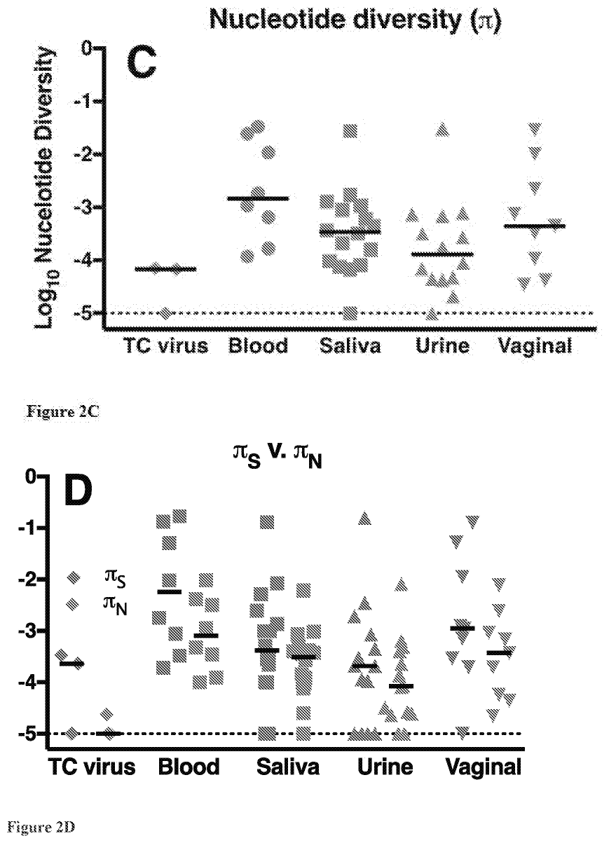

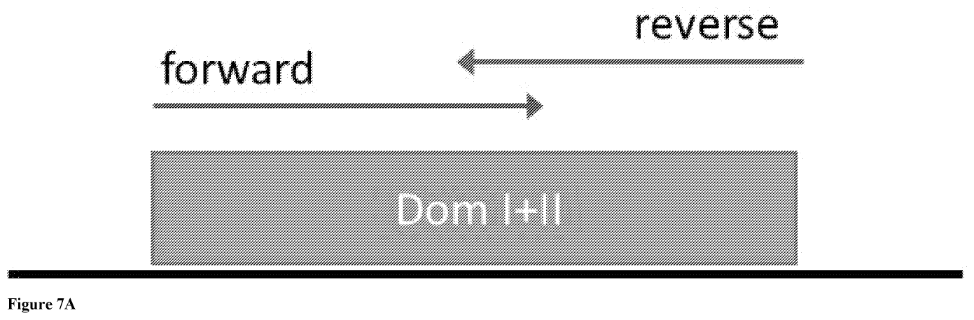

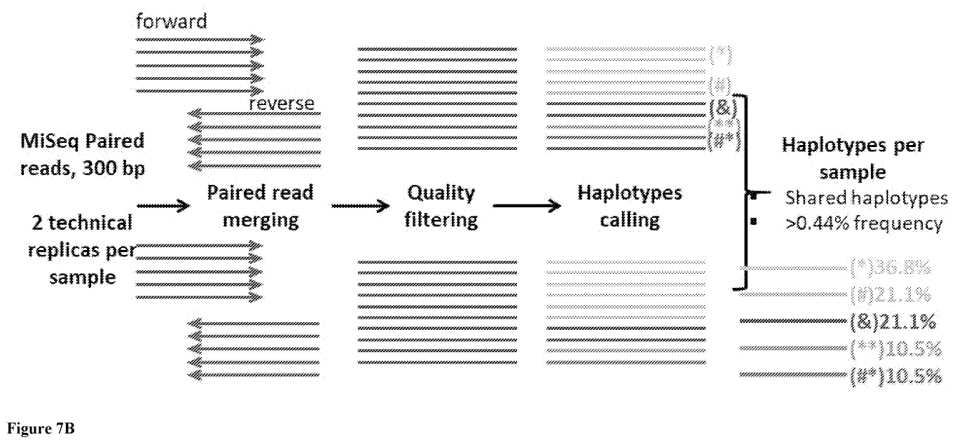

For compartment analysis, all samples (including from chronically HCMV-infected seropositive individuals) were combined. The peak HCMV viral load for each patient in each anatomic compartment was compared (FIG. 2A). As previously reported (23), peak vaginal HCMV shedding was noted to be higher than either plasma viral load (median copies/mL: vaginal=1,705, blood=95; p=0.002, Pairwise Wilcoxon Signed Rank test) and urine shedding (median copies/mL: urine=159; p=0.001, Pairwise Wilcoxon Signed Rank test). There were no statistical differences in the peak number of viral haplotypes identified or peak nucleotide diversity between blood, saliva, urine, or vaginal fluid at either the gB (FIG. 2B,C) or UL130 (FIG. 7C, D) loci. Of note, the nucleotide diversity of plasma HCMV was higher than that of shed HCMV in urine at the gB (median π: blood=1.5×10⁻³, urine=1.3×10⁻⁴) and UL130 (median π: blood=3.8×10⁻⁴, urine=2.1×10⁻⁴) as previously observed (18). Finally, π_{S} exceeding π_{N} was observed in blood (p=0.027, Wilcoxon Signed Rank test), saliva (p=0.008, Wilcoxon Signed Rank test), urine (p=0.011, Wilcoxon Signed Rank test), and vaginal fluid (p=0.010, Wilcoxon Signed Rank test) at the gB locus, as well as in urine at the UL130 locus (p=0.006, Wilcoxon Signed Rank test), again indicating the pervasiveness of purifying selection in these viral populations.

Presence and Persistence of Low-Frequency, Unique HCMV Variants.

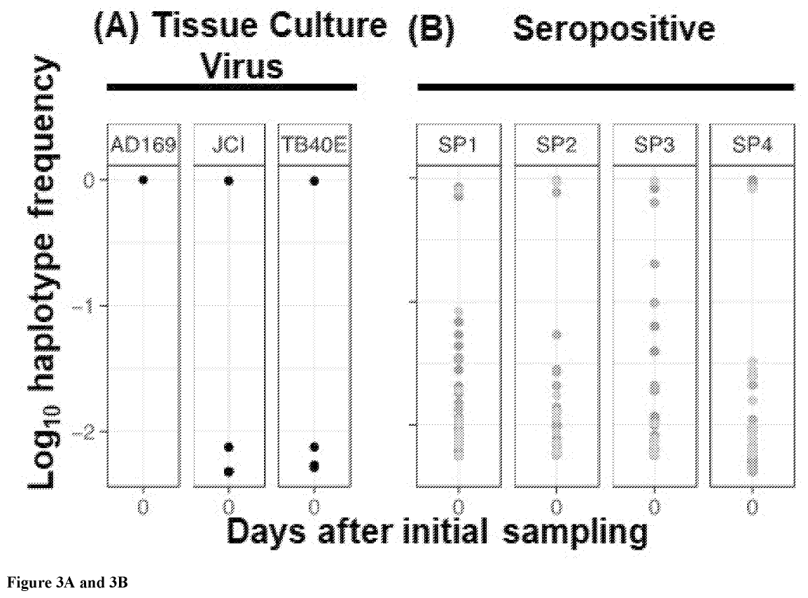

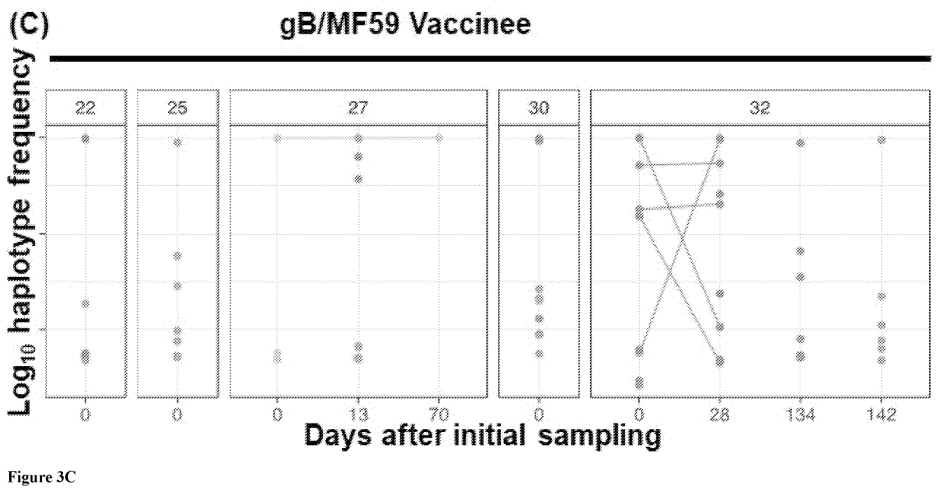

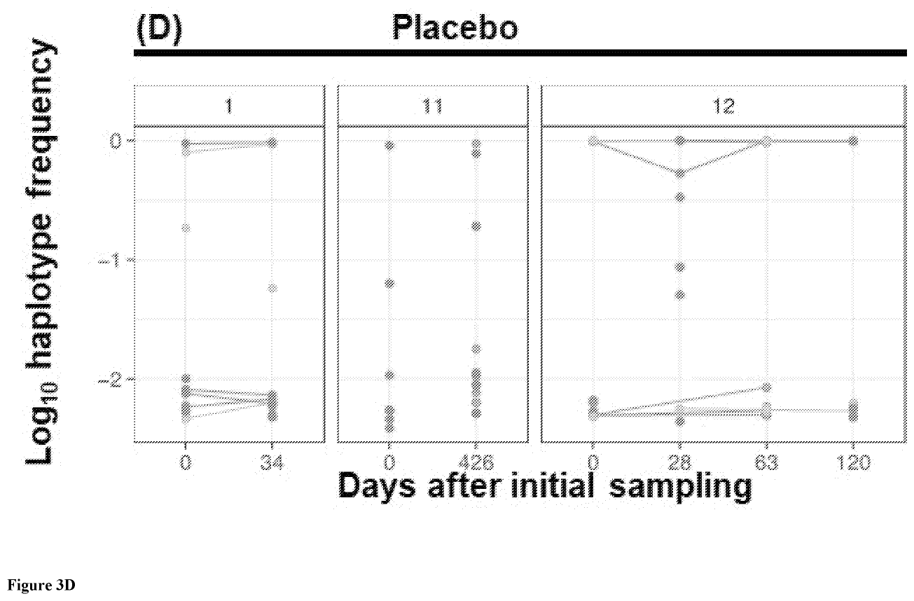

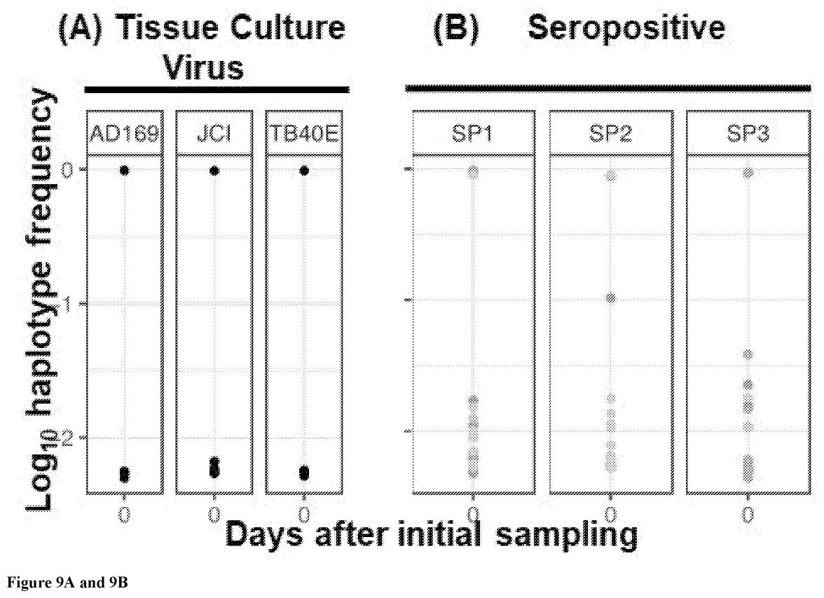

The relative frequency of unique viral haplotypes was identified for tissue culture virus stocks, chronically HCMV-infected individuals, and primary HCMV-infected gB/MF59 vaccines and placebo recipients at the gB (FIG. 3) and UL130 (FIG. 9) genetic loci. For all patients and at both loci, there is typically a single dominant viral variant, with a frequency approaching 100%. Along with this dominant variant, we find multiple minor variants of very low frequency (<1%) that are genetically-distinct from the dominant variant and often persist over time. For example, longitudinal haplotype data for placebo recipients 103 and 455 indicates the persistence of both the dominant variant and the low frequency variants from one time point to the next, indicating that these identified variants are not simply sequencing artifact.

Anatomic Compartmentalization of HCMV Populations in gB/MF59 Vaccines.

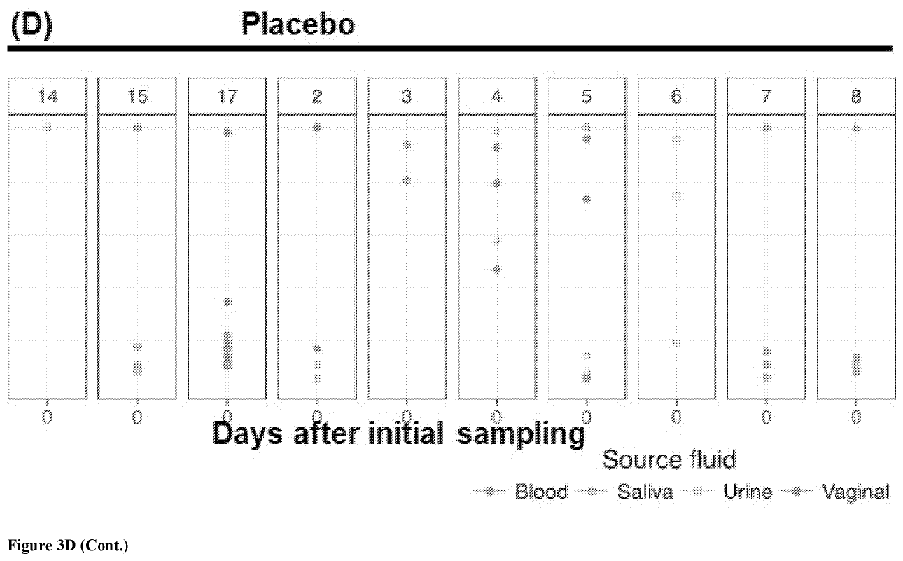

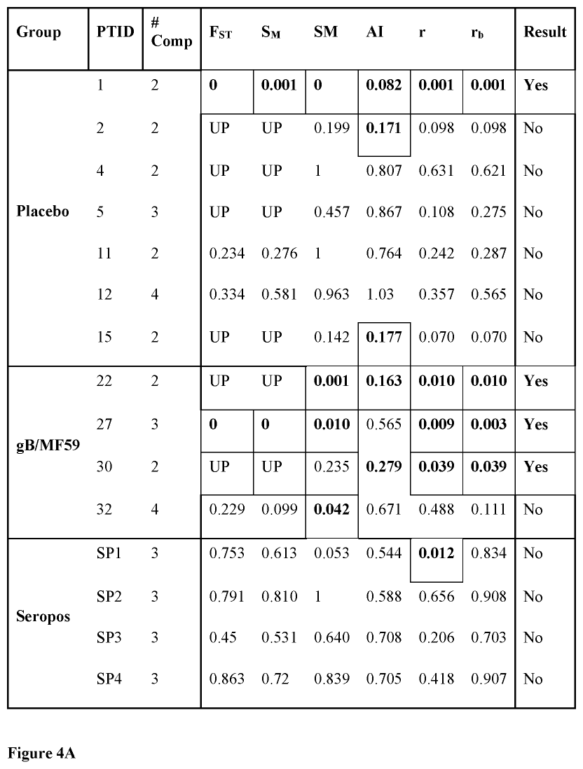

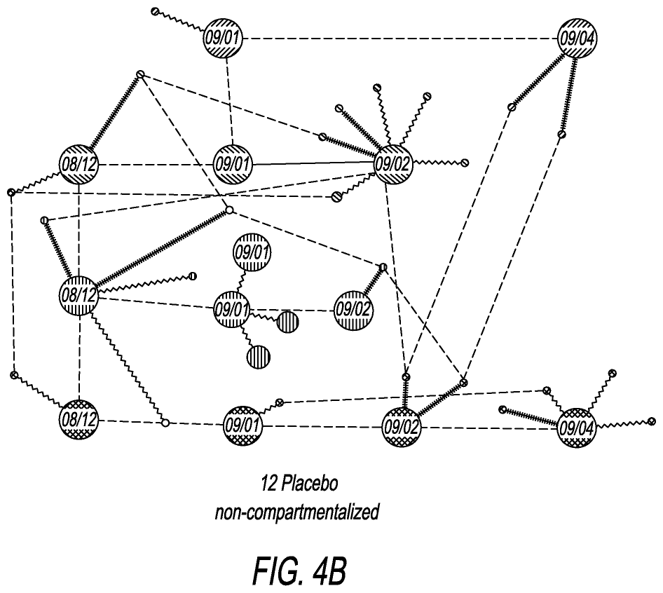

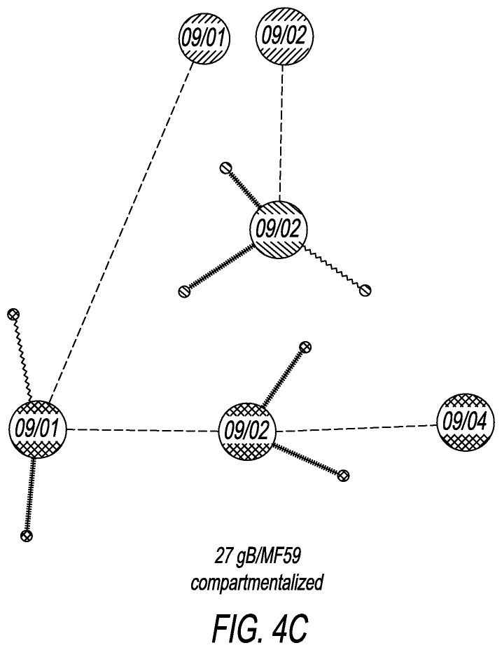

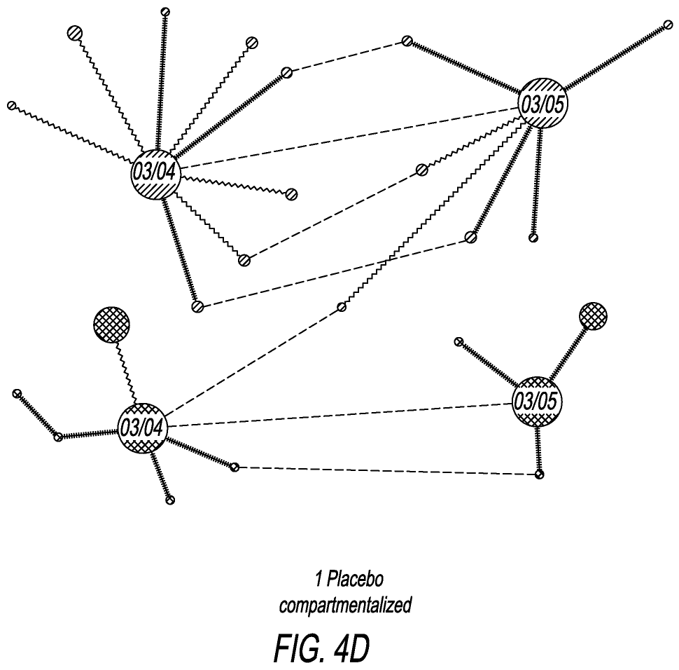

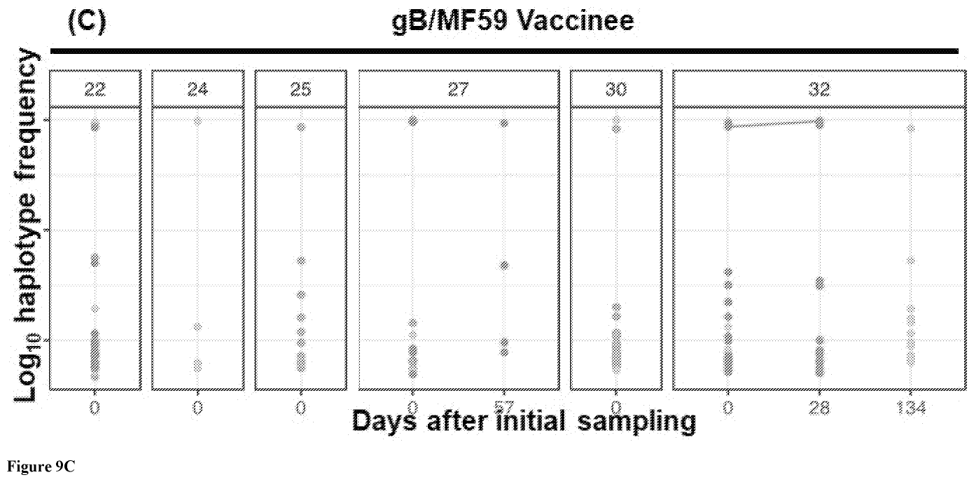

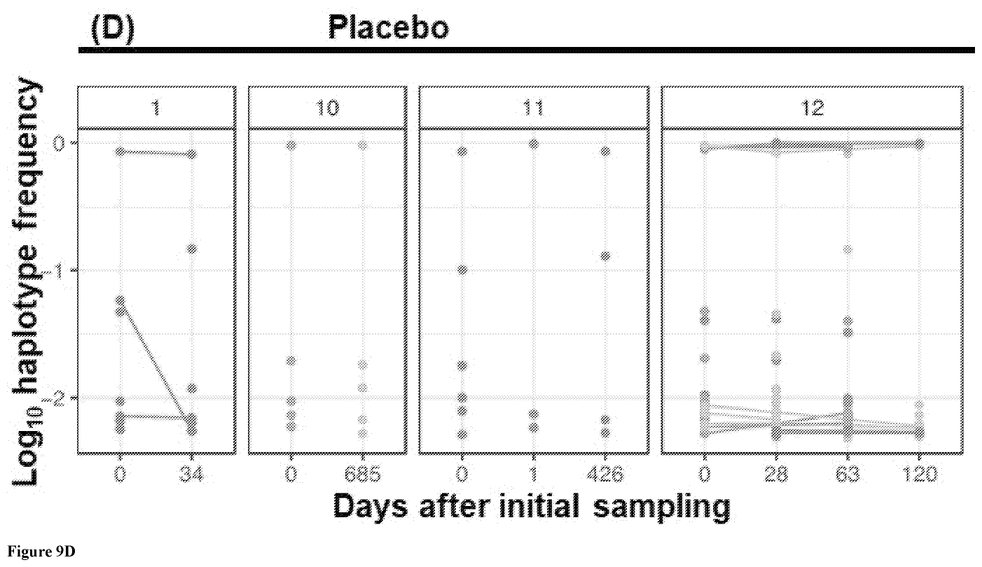

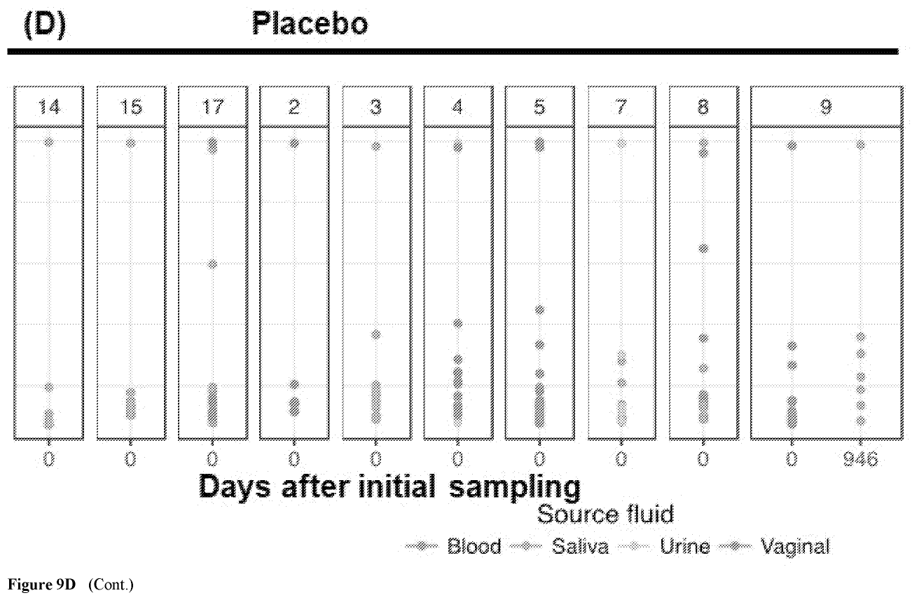

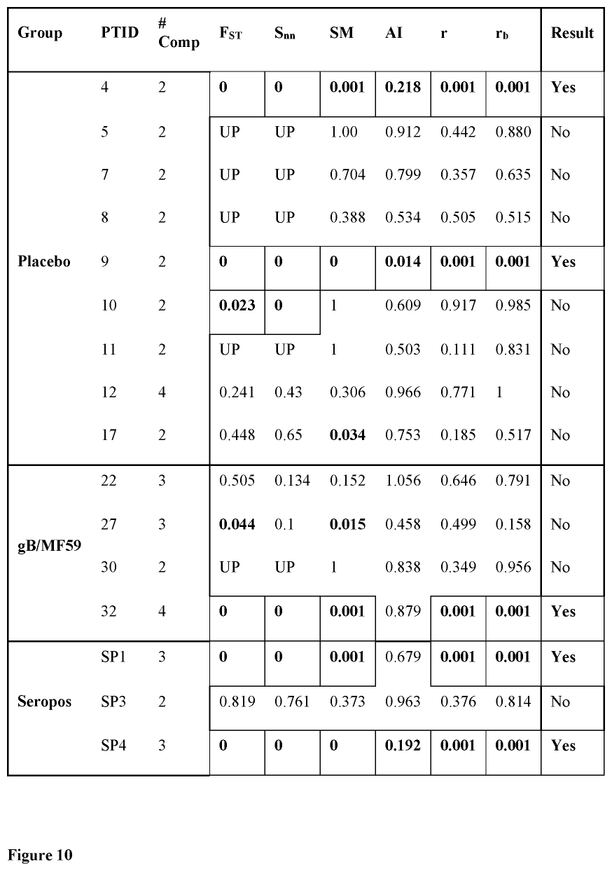

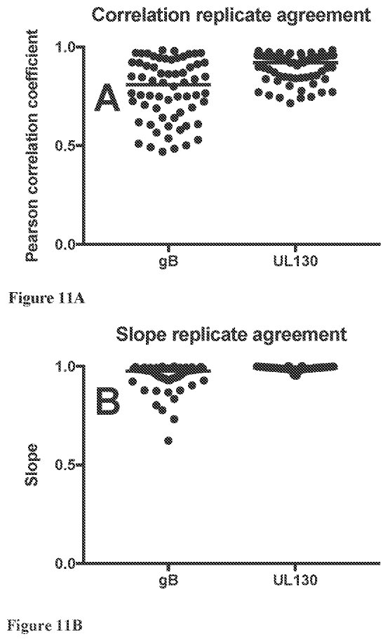

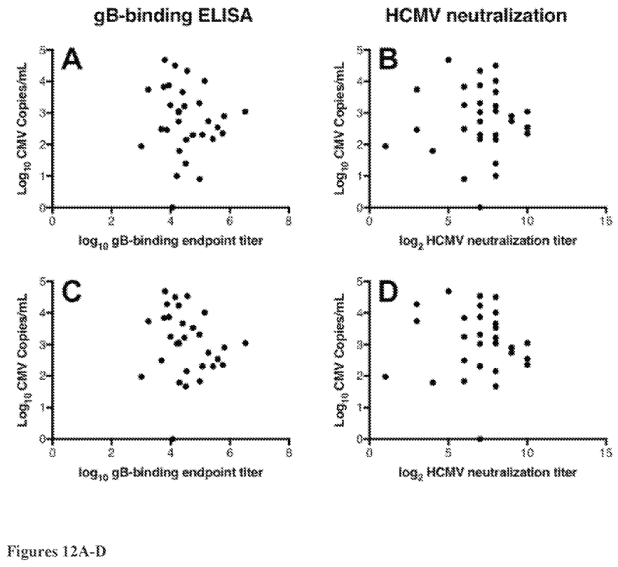

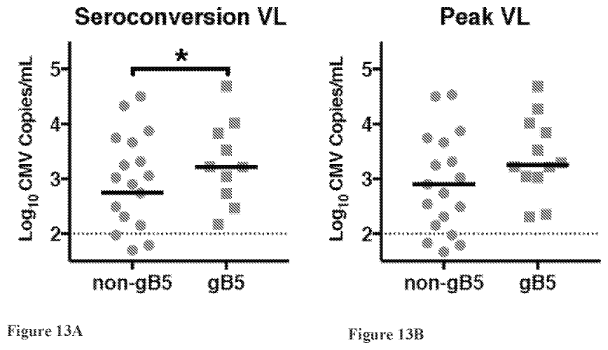

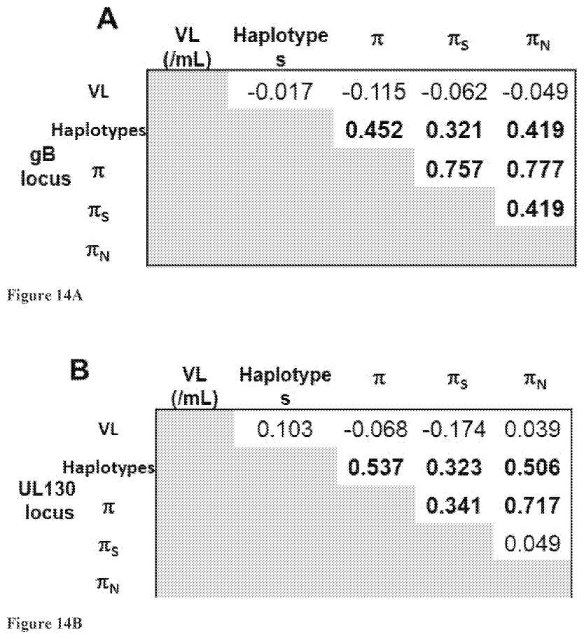

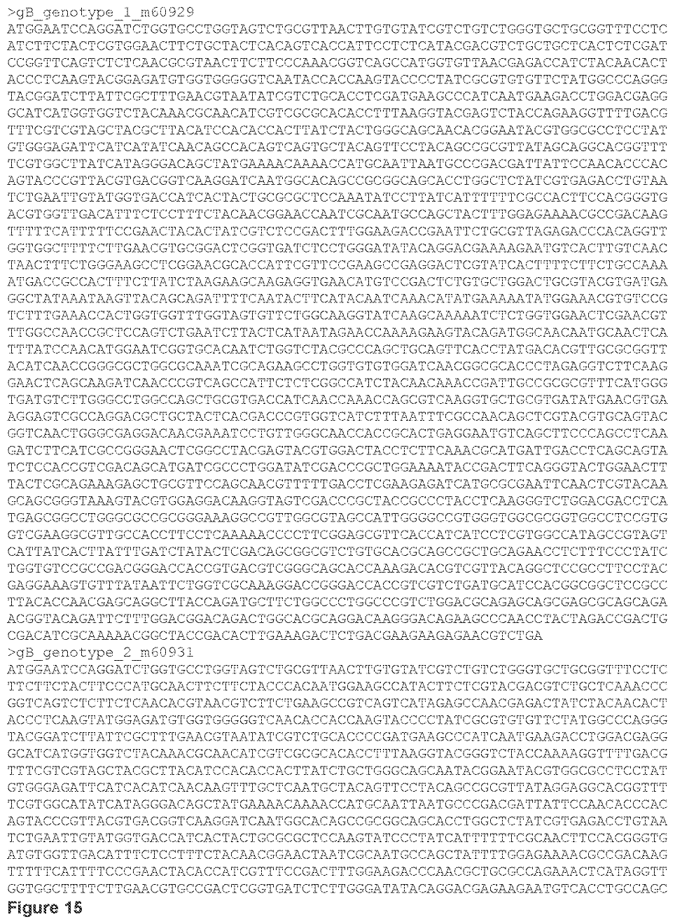

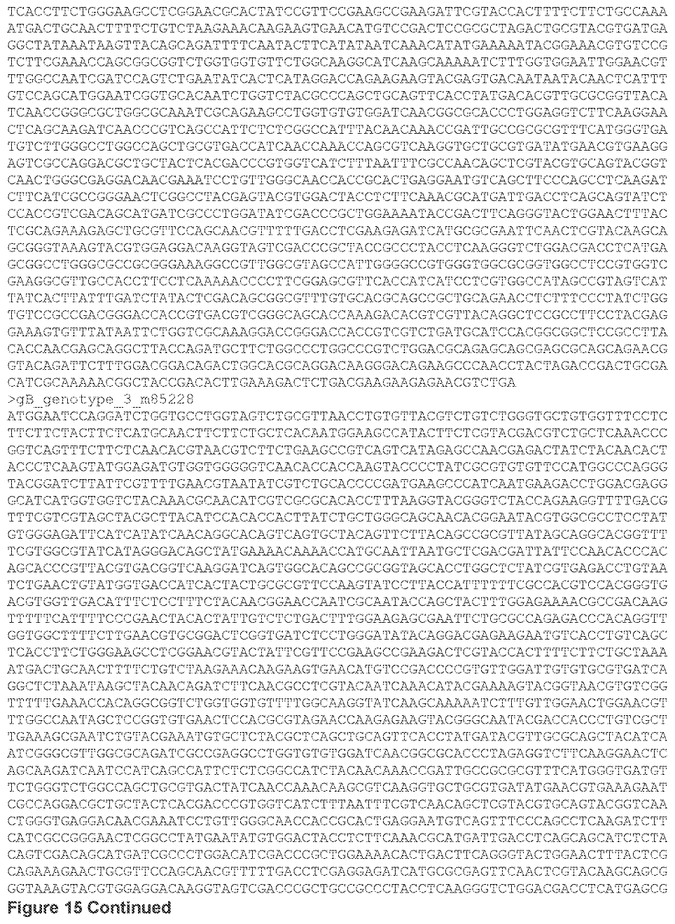

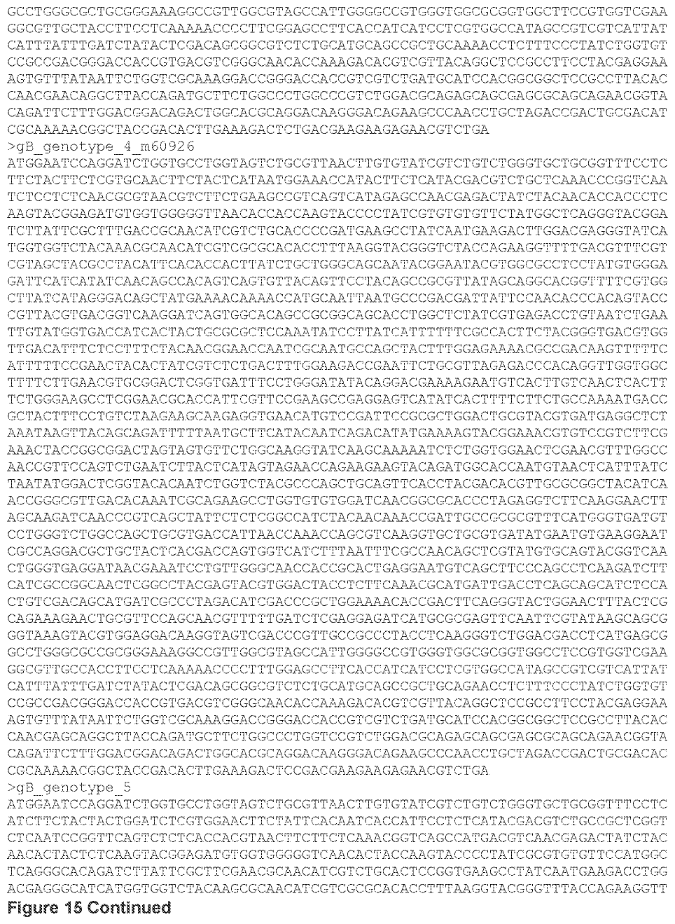

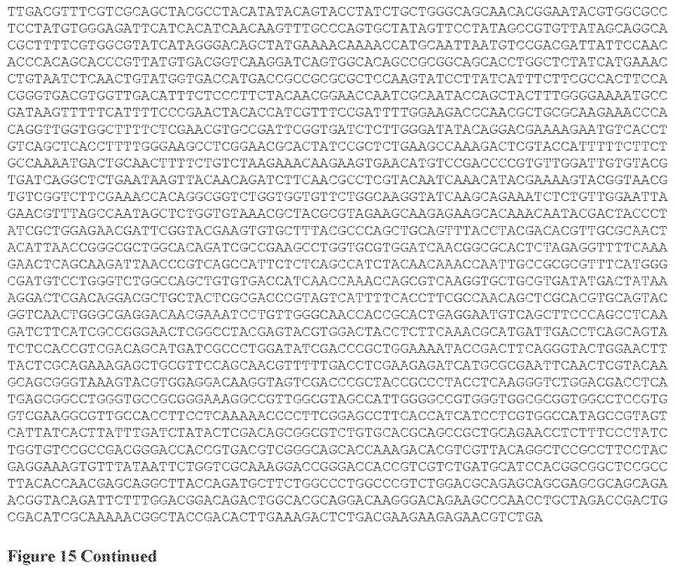

A panel of tests for genetic compartmentalization reliant upon 6 distinct distance and tree-based methods was employed to assess the extent to which viral populations in different anatomic compartments of a single subject constitute distinct populations. Given our definition of compartmentalization based on significant results for at least 3 of 6 tests, anatomic compartmentalization at the gB locus was observed for 1 of 7 placebo recipients, 3 of 4 gB vaccine recipients, and 0 of 4 chronically HCMV-infected individuals (FIG. 4A). Though this frequency of genetic compartmentalization between placebo recipients and gB vaccines was not statistically significant (p=0.088, Fisher's Exact test), there is a trend towards increased compartmentalization in the vaccine group. This same trend was not observed at the UL130 locus, as 2 of 9 placebo recipients, 1 of 4 gB vaccines, and 2 of 3 seropositive individuals exhibited evidence of anatomic virus population compartmentalization (FIG. 10. This is perhaps due to vaccine-mediated immune pressure at the gB, but not UL130 locus. Of interest, no two patients had evidence of genetic compartmentalization at both gB and UL130 loci, perhaps suggesting that these two loci are under independent selection pressures. The pool of gB haplotypes for 3 representative individuals is shown chronologically and separated by anatomic compartment to demonstrate patients either lacking (FIG. 4B) or exhibiting (FIG. 4C, D) evidence of gB variant genetic compartmentalization.

gB Genotype Analysis.

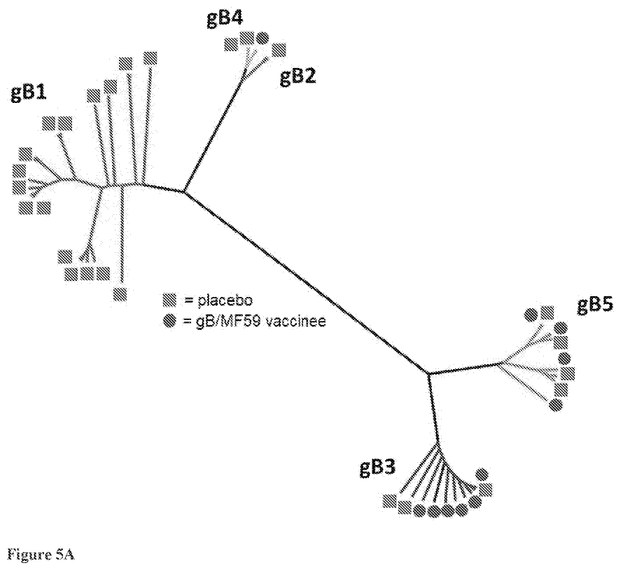

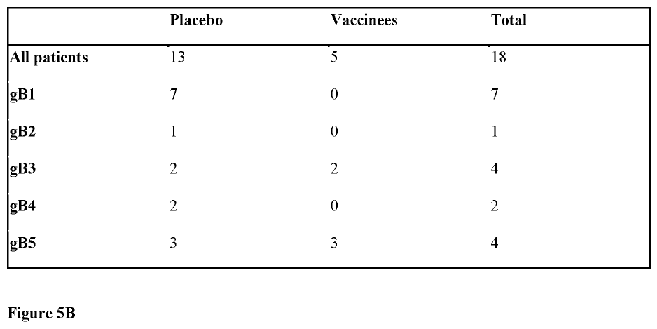

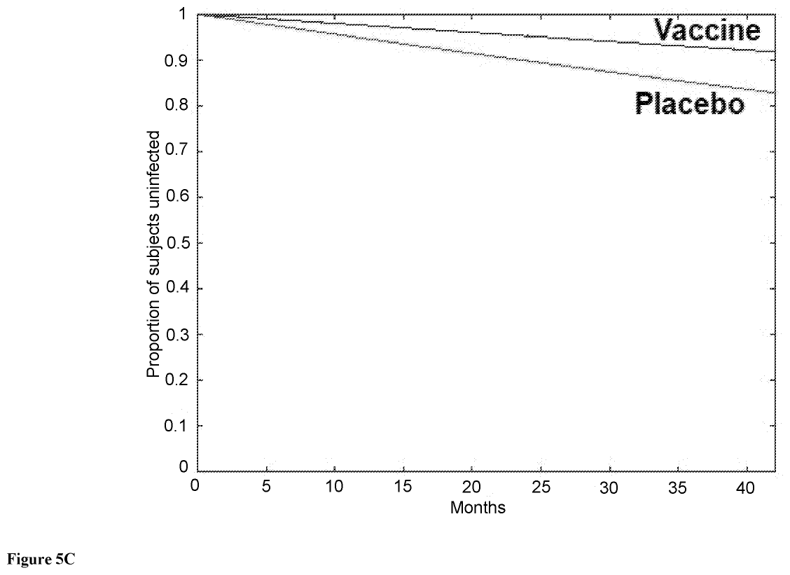

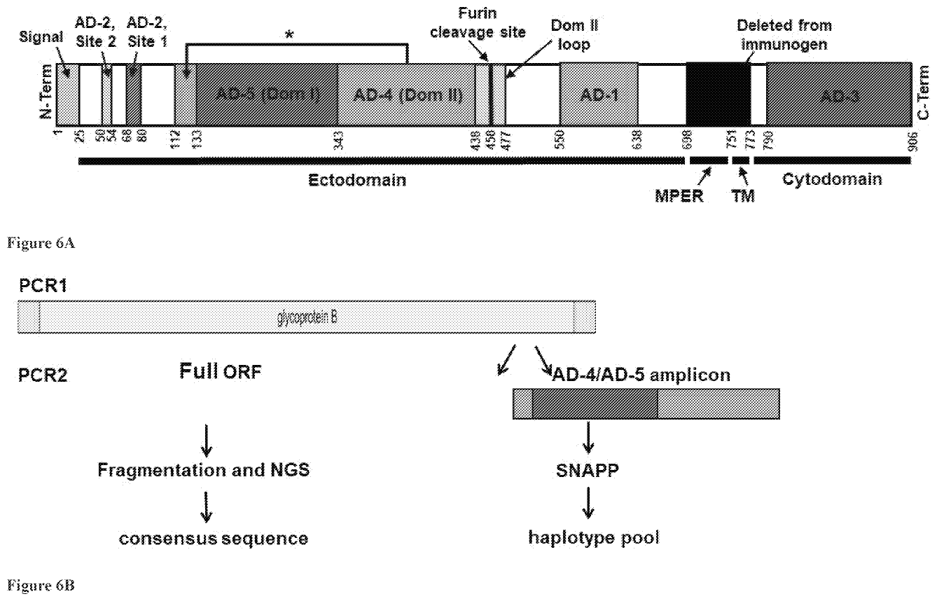

In addition to gB and UL130 SNAPP to define viral haplotypes, the full gB ORF was amplified, fragmented, and sequenced by NGS to identify a gB consensus sequence for each unique sample (FIG. 6. Reassuringly, there was a high level of agreement of the gB genotype identified in primary HCMV-infected women between previously-published Sanger sequencing data (16), full gB ORF NGS, and SNAPP (Table 1). As previously noted by Sanger sequencing, full gB ORF NGS indicated relatively few incidences of mixed infection observed between physiologic compartments or distinct time points. However, on average, the SNAPP technique identified additional, low-frequency viral variants corresponding to diverse gB genotypes, likely only discernable due to the enhanced sensitivity of this technique.

Additionally, we inferred a phylogenetic tree using sequences from the full gB ORF (FIG. 5A). All 5 gB genotypes are clearly distinguishable as unique branches of the tree. We did not observe any preference of specific gB genotypes for any particular anatomic compartment. Yet, we noted a low frequency of gB1 genotype viruses among gB/MF59 vaccines, perhaps suggesting that the gB1 genotype vaccine construct inhibited infection with genetically-similar viruses. Indeed, 7 of 13 placebo recipients acquired a gB1 genotype virus compared to 0 of 5 vaccines (FIG. 5B), though due to small sample size this comparison was not significant (p=0.10. Fisher's Exact test). However, we also noticed there were no gB2 or gB4 genotype viruses acquired by vaccines, although this may be due to chance since these genotypes were not as dominant as gB1 in placebo recipients. Thus, 9 of 13 placebo recipients acquired a gB1/2/4 genotype virus compared to 0 of 5 vaccines (p=0.03, Fishers Exact Test). We sought to investigate whether complete protection from gB1 genotype viruses could have explained the partial vaccine efficacy observed in the gB/MF59 clinical trial by modeling the HCMV force-of-infection. Assuming that gB1 genotype viruses comprise 54% of the circulating virus pool (7 of 13 acquired viruses among placebo recipients) and second that gB vaccines are universally protected against gB1 genotype viruses, we observe that HCMV force-of-infection modeling (FIG. 5C) very closely predicts the results observed in clinical trial (5).

TABLE 1
Distinct gB genotypes detected in various clinical samples from placebo
recipients and gB/MF59 vaccines using different sequencing methodologies.

| | | gB cleavage | Full gB ORF | gB |
|---|---|---|---|---|
| Patient | Group | site Sanger | NGS | SNAPP |
| 1 | Placebo | 4 | 4, 5 | 1, 4, 5 |
| 2 | Placebo | 3 | 3 | 1, 3 |
| 3 | Placebo | 3 | 3 | 1, 3 |
| 4 | Placebo | 1 | 1 | 1, 5 |
| 5 | Placebo | 2 | 2 | 1, 2, 5 |
| 6 | Placebo | 1 | — | 1 |
| 7 | Placebo | 2 | — | 2 |
| 8 | Placebo | 1 | 1 | 1 |
| 9 | Placebo | 1 | 1 | — |
| 10 | Placebo | 1 | 1 | — |
| 11 | Placebo | 1, 4 | 1, 4 | 1, 4 |
| 12 | Placebo | 1 | 1 | 1 |
| 14 | Placebo | 1 | 1 | 1 |
| 15 | Placebo | 5 | 5 | 1, 5 |
| 17 | Placebo | 5 | 5 | 5 |
| 22 | gB/MF59 | 3 | 3 | 1, 3, 5 |
| 25 | gB/MF59 | 1 | 5* | 1 |
| 27 | gB/MF59 | 3 | 3⁺ | 1, 3 |
| 30 | gB/MF59 | 5 | 5 | 1, 5 |
| 32 | gB/MF59 | 5 | 5 | 1, 5 |

### DISCUSSION

Despite the partial efficacy demonstrated by gB/NIF59 vaccination in multiple clinical trials (5-7), there has been little examination of the impact of this vaccine on the in vivo viral populations. In this investigation, we sought to employ the enhanced sensitivity of next-generation sequencing (NGS) technology to delve deeper into the question of whether there are discernable differences between viruses acquired by gB/MF59 vaccines and placebo recipients. The advantage of NGS over more traditional sequencing methodologies is the ability to detect minor viral variants, which contribute to the diversity of the overall viral population (FIG. 3, FIG. 9). We discovered that numerous minor viral haplotypes, exceeding the threshold of PCR and sequencing error, were detectable in nearly all clinical samples tested, which is consistent with results of HCMV whole-genome sequencing that have suggested numerous genetic variants at <1% frequency in the viral population (17-19). Interestingly, seropositive women reliably had more gB haplotypes (FIG. 1E) than acutely-infected vaccinated subjects, indicating a higher number of genetically-distinct viral variants in chronically HCMV-infected individuals. This observation complements previous work demonstrating that recently-seroconverted young women have very low incidence of mixed infection, yet that multiple gB genotypes are almost universally detectable in chronically-HCMV infected individuals (20). Altogether, these data favor a model that mixed infections in healthy individuals result from independent, sequential infection events.

Throughout the study, we identified several indications of vaccine-mediated effects on the viral population. First, peak HCMV shedding in saliva was reduced by an order of magnitude in gB/MF59 vaccines, suggesting that the vaccine responses may limit viral replication in salivary glands. gB/MF59 vaccination is known to elicit high titers of gB-specific IgG, IgA, and SIgA in parotid saliva (24), which may have suppressed HCMV salivary replication and reduced saliva viral shedding. However, there was no difference observed in peak systemic viral load or peak viral load in urine and vaginal fluid between infected vaccines and placebo recipients. Since women were only tested for HCMV acquisition every 3 months and since the time-point of infection and viral load/viral shedding kinetics are unknown, it is possible that sampling limitations may have obscured any differences between groups.

Secondly, we observed that 3 of 4 gB vaccines with viral DNA available from multiple compartments exhibited evidence of viral genetic compartmentalization at the gB locus, in contrast to only 1 of 7 placebo recipients. As has been previously described (16), we observed that the dominant viral variant was identical between anatomic compartments in the majority of subjects. The evaluation of gB-specific compartmentalization was therefore only discernable because of the ability of Short NGS Amplicon Population Profiling (SNAPP) to detect minor viral variants. Our data are consistent with HCMV whole-genome NGS indicating tissue-specific variants, with intrahost variable SNPs at relatively low frequency (17, 18). The mechanism leading to the observed compartmentalization in vaccines is unclear, though it is possible this phenomenon stems from either neutral or positive selection in distinct anatomic compartments. One hypothesis is that systemic gB-specific antibodies prevented unrestricted dissemination of HCMV variants between tissue compartments. Then, this bottleneck might have reduced founder population size and increased the speed and likelihood of stochastic fixation of neutral SNPs and formation of genetically-distinct viral populations (25, 26). Alternatively, it is possible that local factors including cell type and local antibody production/secretion at the site of HCMV replication selected for “more fit” viral variants in each compartment.

Finally, we observed an interesting trend that among viruses for which the full gB ORF was sequenced, 7 of 13 placebo recipients (54%) and 0 of 5 gB/MF59 vaccines (0%) acquired a gB1 genotype virus (p=0.08, Fishers Exact test). If we instead consider the acquisition of genetically-similar gB genotypes gB1, gB2, and gB4 between vaccines and placebo recipients, there is statistically-significant inhibition of acquisition of these genotypically-homologous viruses (gB/MF59=10/13, Placebo=0/5; p=0.03, Fisher's Exact test). Of note, the gB immunogen in this vaccine trial was based on the Towne strain (gB1 genotype prototypic sequence) suggesting the possibility of vaccine genotype-specific protection. Complementarily, we have observed in the same gB/MF59 vaccine cohort that gB-elicited neutralization activity was only detectable against the autologous Towne strain virus, but not heterologous viruses AD169 and TB40/E (Nelson et al. Proc Natl Acad Sci U S A. 2018 Apr. 30. pii: 201800177. doi: 10.1073/pnas.1800177115. [Epub ahead of print] PubMed PMID: 29712861). These observations raise the possibility of gB1 genotype-specific protection against HCMV acquisition based on neutralization of only the autologous virus. Furthermore, as demonstrated by force-of-infection modeling, gB1 genotype-specific protection could explain the 50% partial vaccine efficacy observed in this phase 2 clinical trial. The concept of neutralization breadth has not been explored extensively for HCMV, though several papers have described strain-specific neutralization (27-29). Of note, low frequency gB1 genotype haplotypes were detectable in several vaccines by SNAPP, suggesting there may not have been a true barrier to gB1 genotype acquisition but rather restricted gB1 genotype virus replication.

By far the largest limitation of this study was sample availability. Unfortunately, the scope of our investigation was restricted by: 1) the original sampling timeline employed during the clinical trial, 2) the availability of clinical samples, and 3) the integrity of the DNA more than a decade following DNA extraction. Additionally, as with any study based on PCR amplification and DNA sequencing, there is a potential for primer bias, contamination, and background error to obscure the results. We instituted several measures to increase data integrity. First, primers were designed and validated to prevent amplification bias (21). Additionally, PCR, sequencing, and analysis was completed in duplicate to reduce the likelihood of contamination affecting results. An advantage of this investigation is that we were able to validate our two sequencing methodologies (SNAPP and full gB ORF NGS) by comparing observed gB genotypes with previously published data (16). The gB genotype predicted by Sanger sequencing and NGS sequencing methodologies were identical for 88% of all samples. However, because of the relatively small cohort size and potential for sequencing error, our observed trends certainly merit further investigation.

Nonetheless, this investigation is the first to employ NGS of viral DNA from infected gB/MF59 vaccine and placebo recipients in an attempt to characterize the viral determinants of HCMV acquisition. Our observation of reduced saliva shedding and a high rate of gB sequence compartmentalization in vaccines suggests an impact of gB-elicited antibodies on viral population dynamics. Furthermore, the observation of possible vaccine immunogen genotype (gB1)-specific protection is intriguing, and, when paired with our previous finding that gB/MF59 vaccination elicited neutralization of the autologous (gB1) Towne strain virus but not heterologous virus strains in this same trial (Nelson et al. Proc Natl Acad Sci USA. 2018 Apr. 30. pii: 201800177. doi: 10.1073/pnas.1800177115. PubMed PMID: 29712861), strongly implies that strain-specificity of the immune response may have played a role in vaccine protection. Thus, the impact of including multiple gB genotypes in next-generation HCMV vaccines should be investigated in subsequent studies.

Materials and Methods:

Study population. The study population was comprised of 53 postpartum women who acquired HCMV infection while participating in a phase 2, randomized, double-blind, placebo controlled clinical trial of an HCMV vaccine (16). Clinical trial participants were HCMV-seronegative, healthy postpartum women immunized with gB protein (based on Towne, gB-1 genotype) vaccine (Sanofi Pasteur) with MF59 adjuvant (Novartis) on a 0, 1 and 6 month schedule and were screened for HCMV infection every three months for 3.5 years using an antibody assay which detects seroconversion to CMV proteins other than gB (30). Institutional review board (IRB) approval was obtained from University of Alabama at Birmingham and Johns Hopkins Hospital and all subjects signed an approved consent form. The Duke University Health System determined that analysis of de-identified samples from these cohorts does not constitute human subjects research.

Viral isolation. Subjects with serologic evidence of infection were tested for HCMV in blood, urine, saliva and vaginal swab from one month to 3.5 years after seroconversion. Aliquots of each specimen were stored at −80° C. Fresh urine, saliva and vaginal swab specimens were inoculated into cultures of MRC-5 cells (ATCC) or locally prepared human foreskin fibroblasts. Cultures were checked weekly for 4-6 weeks after inoculation; CMV was identified by its characteristic cytopathic effect. Tissue culture with CMV (primary isolate or first passage) was frozen at −80° C. for later analysis.

DNA extraction and Quantitative PCR. Total genomic DNA was extracted from infected cells using a capture-column kit (Qiagen, Valencia, Calif.). HCMV DNA was extracted from 400 μL of original samples—blood, urine, saliva, or vaginal swab in culture media—using the MagAttract virus mini M48 kit (Qiagen) on Biorobot M48. The quantitative PCR assay is based on amplification of a 151-bp region from the US17 gene (23, 31). As previously reported, the limit of detection is 100 copies/mL (4 copies/well), with a measurable range of 100 to 10⁸ copies per mL.

Short NGS amplicon population profiling (SNAPP). Flow chart detailing the sequencing strategy is shown in FIG. S1. Variable regions approximately 550 base-pairs in length within gB (UL55) and UL130 were amplified in duplicate by a nested PCR using the primers denoted in Table 2. Overhang regions were conjugated to PCR2 primers for subsequent Illumina index primer addition and sequencing: forward primer overhang=5′-TCGTCGGCAGCGTCAGATGTGTATAAGAGACAG-[locus]-3′ and reverse primer overhang=5′-GTCTCGTGGGCTCGGAGATGTGTATAAGAGACAG-[locus]-3′. Template DNA extracted from primary fluids was added to 50 μl of 1× PCR mixture containing 100 nM of each primer, 2 mM MgCl₂, 200 μM each of dNTP mix (Qiagen), and 0.2 U/μl Platinum Taq polymerase. PCR reactions consisted of an initial 2-minute denaturation at 98° C., followed by 35 PCR cycles (98° C. for 10 seconds, 65° C. for 30 seconds, and 72° C. for 30 seconds), and a final 72° C. extension for 10 minutes. Following each amplification step, products were purified using Agencourt AMPure XP beads (Beckman Coulter). Illumina Nextera XT index primers were added by 15 cycles of amplification. The indexed PCR product was run on a 1% agarose gel, then gel-purified using ZR-96 Zymoclean Gel DNA Recovery Kit (Zymogen). The molar amount of each sample was normalized by real-time PCR using the KAPA library amplification kit (KAPA Biosystems). The library of individual amplicons was pooled together, diluted to an end concentration of 14 pM, combined with 20% V3 PhiX (Illumina), and then sequenced on Illumina Miseq using a 600-cycle V3 cartridge (Illumina).

TABLE 2
Primer sequences.

| Primer | Sequence |
|---|---|
| fullgB_PCR1_F | 5′-ACACGCAAGAGACCACGACG-3′ |
| fullgB_PCR1_R | 5-TTGAAAAACATAGCGGACCG-3′ |
| fullgB_PCR2_F | 5′-ATGGAATCCAGGATCTGGTG-3′ |
| fullgB_PCR2_R | 5′-TCAGACGTTCTCTTCTTCGT-3′ |
| gBamp_PCR2_F | 5′-Illumina_overhang-GAAAA |
| | CAAAACCATGCAATT-3′ |
| gBamp_PCR2_R | 5′-Illumina_overhang-GTCGG |
| | ACATGTTCACTICTT-3′ |
| UL130_PCR1_F | 5′-TGGGATGGGTGCAGAAGGT-3′ |
| UL130_PCR1_R | 5′-GGCTTCTGCTTCGTCACCAC-3′ |
| UL130_PCR2_F | 5′-Illumina_overhang-ATCGC |
| | ACCTGAAAAGACACG-3′ |
| UL130_PCR2_R | 5′-Illumina_overhang-CCCCG |
| | CCATGGTCTAAACTG-3′ |

Full glycoprotein B open reading frame PCR and sequencing. Flow chart denoting sequencing strategy is shown in FIG. 6. The full gB open reading frame (ORF) was amplified by nested PCR using the primers denoted in Table 2. Template DNA extracted from primary fluids was added to 50 μl of 1× PCR mixture containing 100 nM of each primer. 2 mM MgCl₂, 200 μM each of dNTP mix (Qiagen), and 0.2 U/μl Platinum Taq polymerase. PCR reactions consisted of an initial 2-minute denaturation at 98° C., followed by 35 PCR cycles (98° C. for 10 seconds, 65° C. for 30 seconds, and 72° C. for 3 minutes), and a final 72° C. extension for 10 minutes. Following each amplification step, products were purified using Agencourt AMPure XP beads (Beckman Coulter). The PCR2 product was run on a 1% agarose gel, then gel extracted using the ZR-96 Zymoclean Gel DNA Recovery Kit (Zymogen). Purified product was tagmented using the Nextera XT library prep kit (Illumina). Subsequently, Nextera XT index primers were added to the tagmented DNA by 15 cycles of amplification. The molar amount of each sample was normalized by real-time PCR using the KAPA library amplification kit (KAPA Biosystems). The library of individual amplicons was pooled together, diluted to an end concentration of 14 pM, combined with 20% V3 PhiX (Illumina), and then sequenced on Illumina Miseq using a 600-cycle V3 cartridge (Illumina).

SNAPP haplotype reconstruction and nucleotide diversity. Data processing flow chart is shown in FIG. 7. First, raw paired-end reads were merged using the PEAR software under default parameters (32). The fused reads were then filtered using the extractor tool from the SeekDeep pipeline (baileylab.umassmed.edu/SeekDeep) (22), filtering sequences according to their length, overall quality scores, and presence of primer sequences. All filtered sequencing reads were included for subsequent haplotype reconstruction using the qluster tool from SeekDeep. This software accounts for possible sequencing errors by collapsing fragments with mismatches at low-quality positions. For each given sample, haplotypes had to be present in both of 2 sample replicates to be confirmed. On average, concordance between the replicates was quite high as assessed by linear regression correlation and slope of the relative frequencies of each haplotype (FIG. 11). Each gB haplotype was assigned to 1 of 5 described gB genotypes by assessing the shortest genetic distance (nucleotide substitutions) between the haplotype and reference gB genotype sequences. Nucleotide diversity (π) was computed as the average distance between each possible pair of sequences (33):

$$π = \frac{{\sum }_{i}^{H} {\sum }_{j \leq i}^{H} {d}_{ij} {f}_{i} {f}_{j}}{L * N ( N - 1 ) / 2}$$

Where L=sequence length in nucleotides for π. N=Total number of reads in sample, dij=Number of nucleotide differences between haplotype i and j, fi=Number of reads belonging to haplotype i, π_{N}S and rrs were calculated as the average dS and dN between pairs of haplotypes weighted by the haplotypes abundance:

$${π}_{S} = \frac{{\sum }_{i}^{H} {\sum }_{j \leq i}^{H} {d}_{{S}_{i j}} {f}_{i} {f}_{j}}{L * N ( N - 1 ) / 2}$$

Where L=sequence length in amino acids for πN, πS, N=Total number of reads in sample, dSij=dS between haplotype i and j sequences, fi=Number of reads belonging to haplotype i. Correlations were performed between various measures of viral population diversity (viral load, number of haplotypes, π, π_{S}, and πi_{N}), and suggest that haplotypes, π, π_{S}, and πi_{N} are somewhat related measures although are not directly equivalent (FIG. S9). Assessment of anatomic compartmentalization of virus populations. A panel of tests using diverse analytical methods is hypothesized to be the most accurate means to infer tissue compartmentalization (34). Thus, we selected six tests employing both distance-based and tree-based algorithms. Wright's measure of population subdivision (F_{ST}, distance-based) compares mean pairwise genetic distance between sequences from the same compartment to that of sequences from the same compartment (35). The nearest-neighbor statistic (Sₙₙ, distance-based) measures how frequently the nearest neighbor to each sequence is in the same or different compartments (36). The Slatkin-Maddison test (SM, tree-based) calculates the minimum number of migration events between compartments, compared to the number of migration events expected in a randomly-distributed population (37). The Simmonds association index (AI, tree-based) examines the complexity of the phylogenetic tree structure (38). Finally, correlation-coefficients (r and r_{b}, tree-based) correlates distances between sequences in a phylogenetic tree with compartment of origin based either on distance between sequences (r) or number of tree branches between sequences (r_{b}). Distance-based tests used the TN93 distance matrix (39), and tree-based methods employed a neighbor-joining algorithm. Distance-based tests were not conducted for patients with fewer than 5 haplotypes per compartment since this is known to produce unreliable results (34). All tests were conducted using HyPhy software (veg.github.io/hyphy-site) (version 2.22), with test statistics estimated from 1000+ permutations. For each test, a p-value of <0.05 or an association index <0.3 was considered statistically significant. Three or more positive test results from these six test statistics was considered strong evidence for compartmentalization.

Phylogenetic trees and genotype assignment. Protein or nucleotide sequences of interest were aligned using the ClustalW algorithm (40) in Mega (version 6.06) (41). A neighbor-joining tree was constructed using the Los Alamos National Labs “neighbor treemaker” (accessed at Los Alamos Database hiv.lanl.gov/components/sequence/HIV/treemaker/treemaker.html), then the tree was plotted in FigTree (version 1.4.3). Full gB ORF sequences were assigned to gB genotypes based on phylogenetic proximity to reference gB sequences from GenBank. Because of sample limitation, if the full gB assigned genotype did not match the genotype assigned to SNAPP amplicons and/or previously published Sanger sequencing data (16), these sequences were omitted from the phylogenetic analysis.

Force-of-infection modeling. The cohort of women in the postpartum gB/MF59 vaccine trial were predominantly African-American (>70%) (5), and thus we utilized an HCMV force-of-infection estimate for non-Hispanic, African-American individuals of 5.7 per 100 persons (42). Additionally, we made the assumption that gB1 genotype viruses comprise 54% of the circulating virus pool, based on 6 of 11 placebo recipients acquiring gB1 genotype viruses. Finally, we hypothesized that gB1-vaccinated individuals were universally protected against all circulating gB1 genotype viruses. Modeling was done using Matlab, and source code is included in the supplementary material.

### REFERENCES FOR EXAMPLE 1

1. Dollard S C, Grosse S D, Ross D S. New estimates of the prevalence of neurological and sensory sequelae and mortality associated with congenital cytomegalovirus infection. Rev Med Virol. 2007; 17(5):355-63.
2. Manicklal S, Emery V C, Lazzarotto T, Boppana S B, Gupta R K. The “silent” global burden of congenital cytomegalovirus. Clin Microbiol Rev. 2013:26(1):86-102.
3. Ramanan P, Razonable R R. Cytomegalovirus infections in solid organ transplantation: a review. Infect Chemother. 2013:45(3):260-71.
4. In: Stratton K R, Durch J S, Lawrence R S, editors. Vaccines for the 21st Century: A Tool for Decisionmaking. The National Academies Collection: Reports funded by National Institutes of Health. Washington (DC)2000.
5. Pass R F, Zhang C. Evans A, Simpson T, Andrews W, Huang M L, et al. Vaccine prevention of maternal cytomegalovirus infection. The New England journal of medicine. 2009; 360(12):1191-9.
6. Bernstein D I, Munoz F M, Callahan S T, Rupp R, Wootton S H, Edwards K M, et al. Safety and efficacy of a cytomegalovirus glycoprotein B (gB) vaccine in adolescent girls: A randomized clinical trial. Vaccine. 2016; 34(3):313-9.
7. Griffiths P D, Stanton A, McCarrell E, Smith C, Osman M, Harber M. et al. Cytomegalovirus glycoprotein-B vaccine with MF59 adjuvant in transplant recipients: a phase 2 randomised placebo-controlled trial. Lancet. 2011; 377(9773):1256-63.
8. Dolan A. Cunningham C, Hector R D, Hassan-Walker A F, Lee L, Addison C. et al. Genetic content of wild-type human cytomegalovirus. The Journal of general virology. 2004; 85(Pt 5):1301-12.
9. Davison A J, Dolan A, Akter P, Addison C, Dargan D J, Alcendor D J, et al. The human cytomegalovirus genome revisited: comparison with the chimpanzee cytomegalovirus genome. J Gen Virol. 2003; 84(Pt 1):17-28.
10. Huang E S, Huong S M, Tegtmeier G E, Alford C. Cytomegalovirus: genetic variation of viral genomes. Annals of the New York Academy of Sciences. 1980,354:33246.
11. Bradley A J, Kovacs I J, Gatherer D, Dargan D J, Alkharsah K R, Chan P K, et al. Genotypic analysis of two hypervariable human cytomegalovirus genes. J Med Virol. 2008; 80(9):1615-23.
12. Coaquette A, Bourgeois A, Dirand C, Varin A. Chen W, Herbein G. Mixed cytomegalovirus glycoprotein B genotypes in immunocompromised patients. Clin Infect Dis. 2004:39(2):155-61.
13. Peek R, Verbraak F, Bruinenberg M, Van der Lelij A, Van den Horn G, Kijlstra A. Cytomegalovirus glycoprotein B genotyping in ocular fluids and blood of AIDS patients with cytomegalovirus retinitis. Invest Ophthalmol Vis Sci. 1998; 39(7):1183-7.
14. Sowmya P, Madhavan H N. Analysis of mixed infections by multiple genotypes of human cytomegalovirus in immunocompromised patients. J Med Virol. 2009:81(5):861-9.
15. Hassan-Walker A F, Okwuadi S, Lee L, Griffiths P D, Emery V C. Sequence variability of the alpha-chemokine UL146 from clinical strains of human cytomegalovirus. J Med Virol. 2004; 74(4):573-9.
16. Murthy S, Hayward G S, Wheelan S. Forman M S, Ahn J H, Pass R F, et al. Detection of a single identical cytomegalovirus (CMV) strain in recently seroconverted young women. PloS one. 2011; 6(1):e15949.
17. Renzette N, Bhattacharjee B, Jensen J D, Gibson L, Kowalik T F. Extensive genome-wide variability of human cytomegalovirus in congenitally infected infants. PLoS pathogens. 2011; 7(5):e1001344.
18. Renzette N. Gibson L, Bhattacharjee B, Fisher D, Schleiss M R, Jensen J D, et al. Rapid intrahost evolution of human cytomegalovirus is shaped by demography and positive selection. PLoS Genet. 2013; 9(9):e1003735.
19. Renzette N, Pokalyuk C, Gibson L, Bhattacharjee B. Schleiss M R, Hamprecht K, et al. Limits and patterns of cytomegalovirus genomic diversity in humans. Proceedings of the National Academy of Sciences of the United States of America. 2015; 112(30):E4120-8.
20. Novak Z, Ross S A, Patro R K, Pati S K, Kumbla R A, Brice S, et al. Cytomegalovirus strain diversity in seropositive women. J Clin Microbiol. 2008:46(3):882-6.
21. Nelson C S, Cruz D V, Tran D, Bialas K M, Stamper L. Wu H, et al. Preexisting antibodies can protect against congenital cytomegalovirus infection in monkeys. JCI Insight. 2017; 2(13).
22. Hathaway N J, Parobek C M, Juliano J J, Bailey J A. SeekDeep: single-base resolution de novo clustering for amplicon deep sequencing. Nucleic Acids Res. 2017.
23. Forman M S, Vaidya D, Bolorunduro O, Diener-West M, Pass R F, Arav-Boger R. Cytomegalovirus Kinetics Following Primary Infection in Healthy Women. The Journal of infectious diseases. 2017:215(10):1523-6.
24. Wang J B, Adler S P, Hempfling S. Burke R L, Duliege A M, Starr S E, et al. Mucosal antibodies to human cytomegalovirus glycoprotein B occur following both natural infection and immunization with human cytomegalovirus vaccines. The Journal of infectious diseases. 1996; 174(2):387-92.
25. Kimura M. On the probability of fixation of mutant genes in a population. Genetics. 1962:47:713-9.
26. Kimura M, Ohta T. The Average Number of Generations until Fixation of a Mutant Gene in a Finite Population. Genetics. 1969; 61(3):763-71.
27. Klein M, Schoppel K, Amvrossiadis N, Mach M. Strain-specific neutralization of human cytomegalovirus isolates by human sera. Journal of virology. 1999; 73(2):878-86.
28. Urban M, Britt W, Mach M. The dominant linear neutralizing antibody-binding site of glycoprotein gp86 of human cytomegalovirus is strain specific. Journal of virology. 1992; 66(3):1303-11.
29. Meyer H, Sundqvist V A, Pereira L, Mach M. Glycoprotein gp116 of human cytomegalovirus contains epitopes for strain-common and strain-specific antibodies. J Gen Virol. 1992; 73 (Pt 9):2375-83.
30. Dal Monte P, Pignatelli S, Mach M, Landini M P. The product of human cytomegalovirus UL73 is a new polymorphic structural glycoprotein (gpUL73). J Hum Virol. 2001; 4(1):26-34.
31. Tanaka Y, Kanda Y, Kami M, Mori S, Hamaki T, Kusumi E, et al. Monitoring cytomegalovirus infection by antigenemia assay and two distinct plasma real-time PCR methods after hematopoietic stem cell transplantation. Bone Marrow Transplant. 2002; 30(5):315-9.
32. Zhang J, Kobert K, Flouri T, Stamatakis A. PEAR: a fast and accurate Illumina Paired-End reAd mergeR. Bioinformatics. 2014; 30(5):614-20.
33. Nelson C W, Hughes A L. Within-host nucleotide diversity of virus populations: insights from next-generation sequencing. Infect Genet Evol. 2015:30:1-7.
34. Zarate S. Pond S L, Shapshak P, Frost S D. Comparative study of methods for detecting sequence compartmentalization in human immunodeficiency virus type 1. Journal of virology. 2007; 81(12):6643-51.
35. Hudson R R, Slatkin M, Maddison W P. Estimation of levels of gene flow from DNA sequence data. Genetics. 1992; 132(2):583-9.
36. Hudson R R. A new statistic for detecting genetic differentiation. Genetics. 2000:155(4):2011-4.
37. Slatkin M, Maddison W P. A cladistic measure of gene flow inferred from the phylogenies of alleles. Genetics. 1989; 123(3):603-13.
38. Wang T H, Donaldson Y K, Brettle R P, Bell J E, Simmonds P. Identification of shared populations of human immunodeficiency virus type 1 infecting microglia and tissue macrophages outside the central nervous system. Journal of virology. 2001:75(23):11686-99.
39. Tamura K, Nei M. Estimation of the number of nucleotide substitutions in the control region of mitochondrial DNA in humans and chimpanzees. Mol Biol Evol. 1993:10(3):512-26.
40. Thompson J D, Gibson T J, Higgins D G. Multiple sequence alignment using ClustalW and ClustalX. Curr Protoc Bioinformatics. 2002; Chapter 2:Unit 2 3.
41. Kumar S, Nei M, Dudley J, Tamura K. MEGA: a biologist-centric software for evolutionary analysis of DNA and protein sequences. Brief Bioinform. 2008; 9(4):299-306.
42. Colugnati F A, Staras S A, Dollard S C, Cannon M J. Incidence of cytomegalovirus infection among the general population and pregnant women in the United States. BMC Infect Dis. 2007; 7:71.

### Example 2

Animal Studies

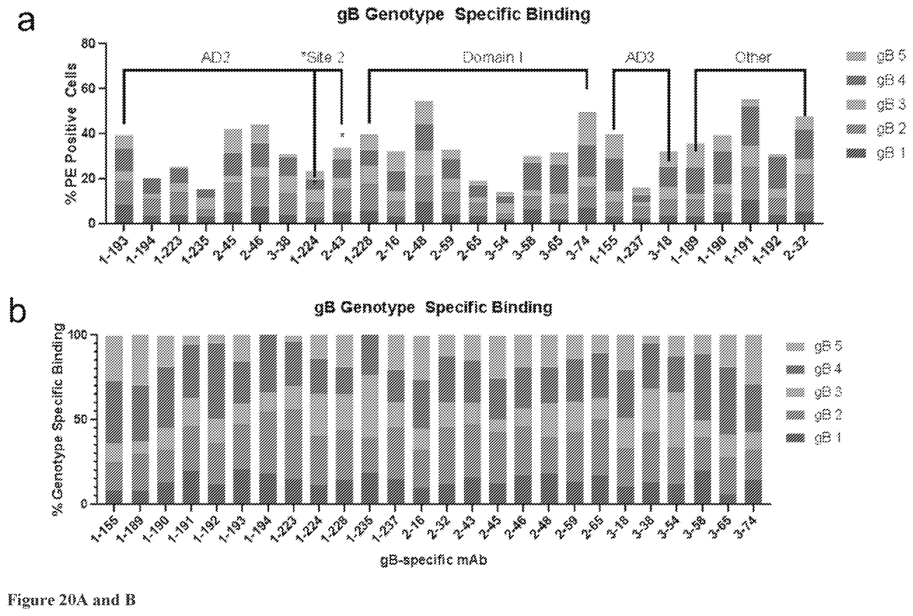

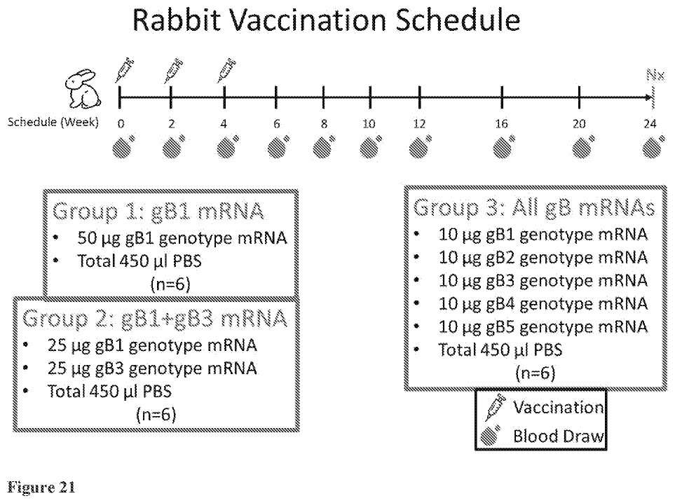

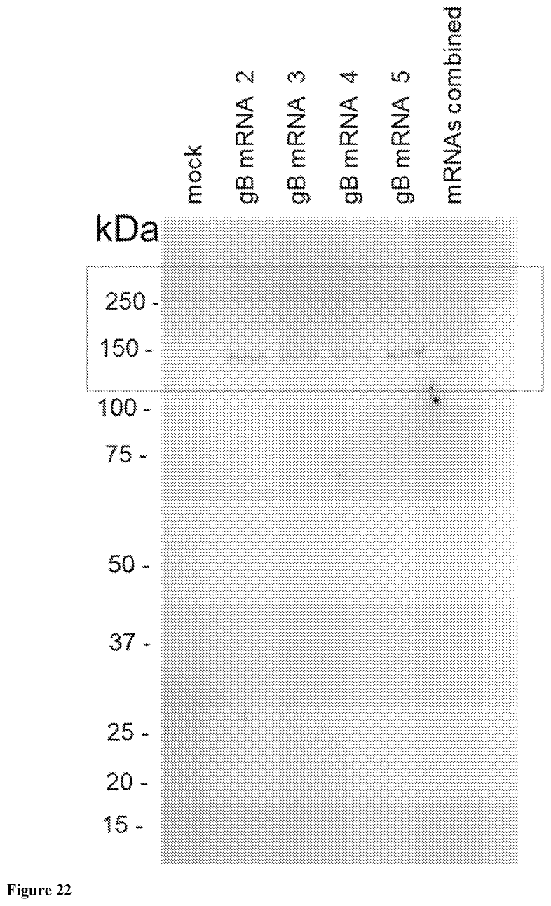

FIG. 21 shows a non-limiting example of an animal study to examine vaccination with multiple gB genotypes.

Animal studies could assess the strength and breadth of immune responses, types of antibodies responses, protection or any other suitable determination that measures responses and/or efficacy.

Additional animal models, such as non-human primates or guinea pigs, could be used to assess whether different CMV genotypes achieve more breadth in the vaccine response, and/or improved responses such as neutralization, protection, ect.

What is claimed is:

1. A multivalent composition comprising at least two antigenic human cytomegalovirus (hCMV) polypeptides or nucleic acids or a portion thereof, wherein the antigens or a portion thereof are of at least two different genotypes.

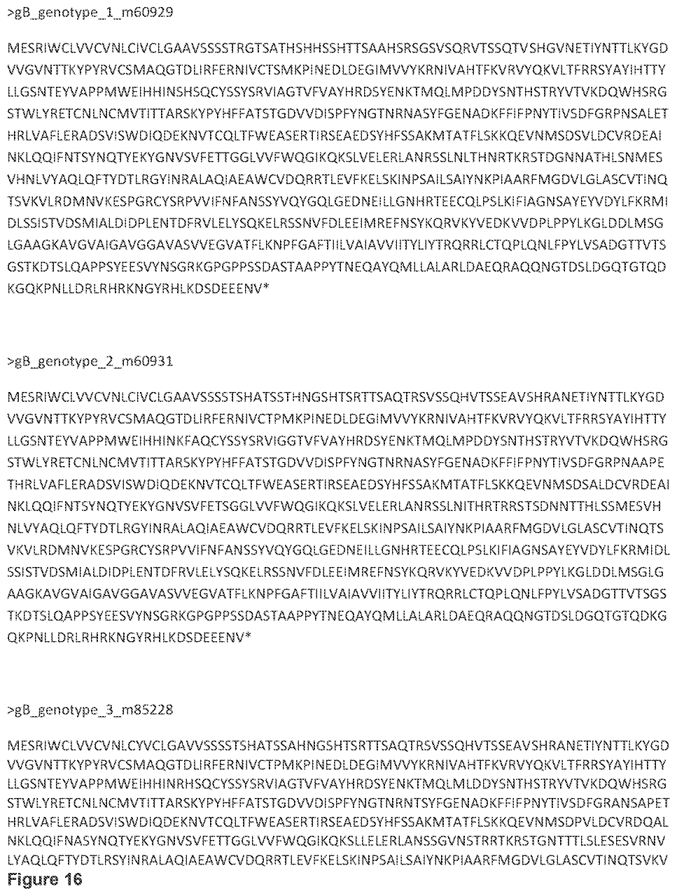

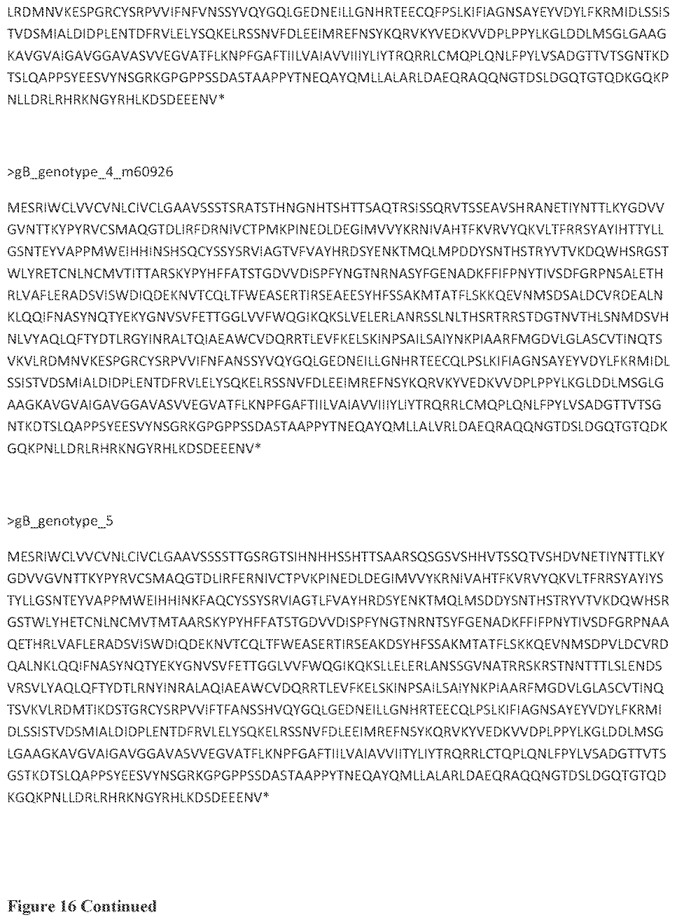

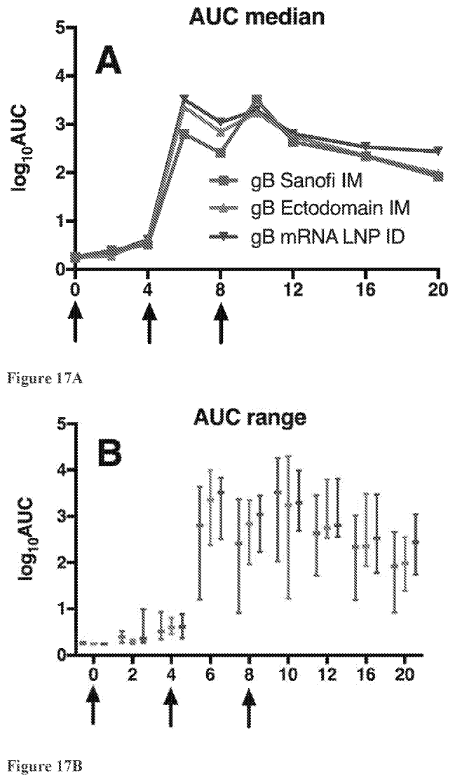

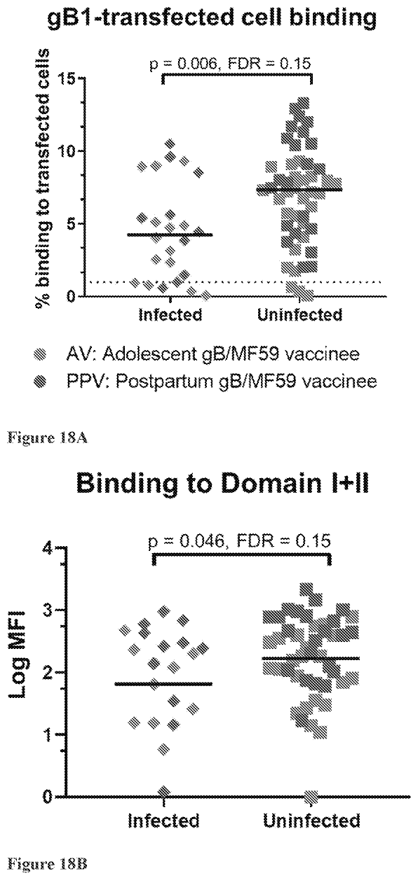

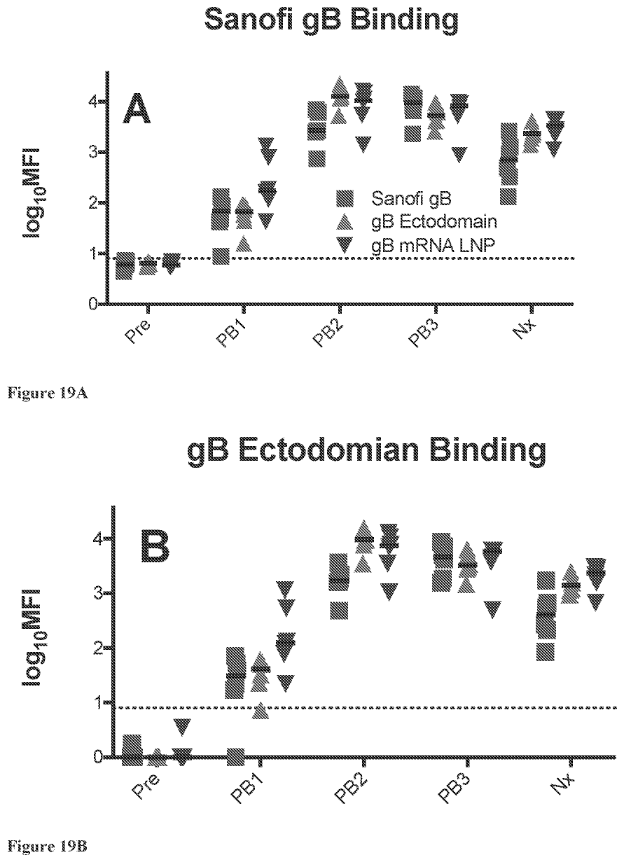

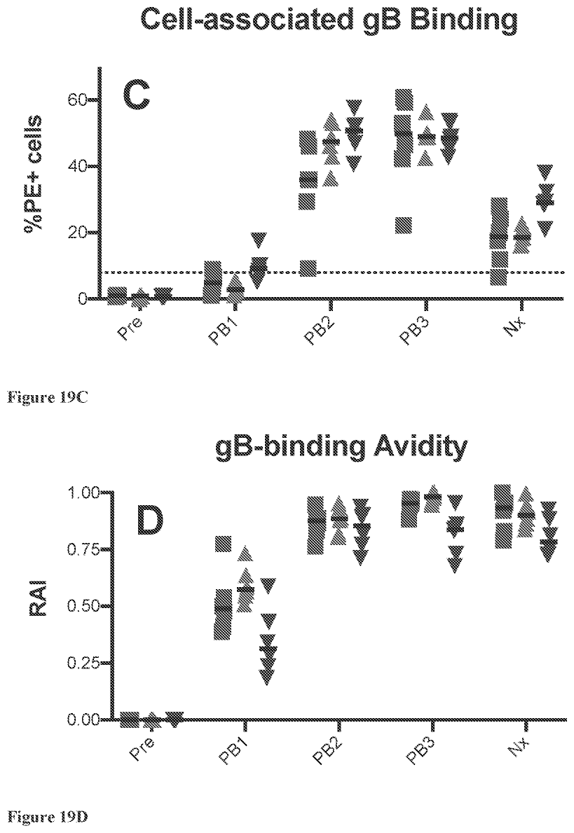

2. A composition comprising hCMV gB5 polypeptide or nucleic acid sequence (of FIG. 16).

3. A multivalent composition comprising at least two hCMV gB polypeptides or nucleic acids encoding gB antigens or a portion thereof, wherein the gB antigens or a portion thereof are of at least two different genotypes, at least three different genotypes, at least four different genotypes, or at least five different genotypes, wherein the genotypes are gB1, gB2, gB3, gB4, or gB5.

4. The composition of claim 3 wherein the at least two different genotypes are: hCMV gB1 and hCMV gB2, hCMV gB1 and hCMV gB3, hCMV gB1 and hCMV gB4, hCMV gB1 and hCMV gB5, hCMV gB2 and hCMV gB3, hCMV gB2 and hCMV gB4, hCMV gB2 and hCMV gB5, hCMV gB3 and hCMV gB4, hCMV gB3 and hCMV gB5, hCMV gB4 and hCMV gB5, or any combination thereof.

5. The composition of claim 3 wherein the at least three different genotypes are: hCMV gB1, hCMV gB2 and hCMV gB3; hCMV gB1, hCMV gB2 and hCMV gB4; hCMV gB1, hCMV gB2 and hCMV gB5; hCMV gB2, hCMV gB3 and hCMV gB4; hCMV gB2, hCMV gB3 and hCMV gB5; hCMV gB3, hCMV gB4 and hCMV gB5, or any combination thereof.

6. The composition of claim 3 wherein the at least four different genotypes are: hCMV gB1, hCMV gB2, hCMV gB3 and hCMV gB4; hCMV gB1, hCMV gB2, hCMV gB3 and hCMV gB5; hCMV gB2, hCMV gB3, hCMV gB4 and hCMV gB5; hCMV gB1, hCMV gB3, hCMV gB4 and hCMV gB5, or any combination thereof.

7. The composition of claim 3 wherein the at least five different genotypes are hCMV gB1, hCMV gB2, hCMV gB3, hCMV gB4 and hCMV gB5.

8. The composition of any one of claim 3-7 wherein the nucleic acid is mRNA.

9. A composition comprising at least one nucleic acid encoding at least one hCMV antigen of at least two different genotypes, at least three different genotypes, at least four different genotypes, or at least five different genotypes, wherein the antigen is gB and the genotypes are gB1, gB2, gB2, gB3, gB4, or gB5, and wherein the nucleic acid is formulated in at least one lipid nanoparticle (LNP).

10. A vector comprising a nucleic acid encoding any one of the antigens of the invention.

11. A host cell comprising a nucleic acid encoding any one of the antigens of the invention.

12. A cell culture comprising any of the host cells of the invention.

13. A method of inducing an immune response against hCMV comprising administering to a subject in need thereof a composition of the invention.

14. Methods of inducing immune responses using the compositions of the invention.
